(19) United States
(12) Patent Application Publication
(10) Pub. No.: US 2015/0011569 A1
(43) Pub. Date: Jan. 8, 2015

(54) NOVEL P13K p110 INHIBITORS AND METHODS OF USE THEREOF

(71) Applicants: PHILADELPHIA HEALTH & EDUCATION CORPORATION d/b/a Drexel University College of Medicine, Philadelphia, PA (US); INSTITUTE FOR HEPATITIS AND VIRUS RESEARCH, Doylestwon, PA (US)

(72) Inventors: Peter D. Katsikis, Merion Station, PA (US); William A. Kinney, Newtown, PA (US); Harold R. Almond, Maple Glen, PA (US); Noshena Khan, Morrisville, PA (US)

(21) Appl. No.: 14/364,751

(22) PCT Filed: Dec. 14, 2012
(86) PCT No.: PCT/US12/69757

§ 371 Date: Jun. 12, 2014

Related U.S. Application Data
(60) Provisional application No. 61/576,178, filed on Dec. 15, 2011.
(60) Provisional application No. 61/584,565, filed on Jan. 9, 2012.

(51) Int. Cl. A61K 31/52; A61K 45/06; C07D 473/34

(52) U.S. Cl. CPC A61K 31/52; C07D 473/34; A61K 45/06
USPC 514/263.22; 544/277; 435/375

(57) ABSTRACT

The invention includes compositions that regulated PI3K p110 delta and are useful as an anti-viral therapy. The invention includes a method of inhibiting p110 delta, a component of PI3K p110 delta signaling pathway, or any combination thereof in a cell as an anti-viral therapeutic approach for treating a viral infection, for example influenza. The invention includes a method of modulating PI3K p110 delta in a cell infected with a virus by contacting the cell with an effective amount of a composition comprising an inhibitor of PI3K p110 delta.

### CROSS-REFERENCE TO RELATED APPLICATION

The present application claims priority to U.S. Provisional Application No. 61/576,178, filed Dec. 15, 2011, and No. 61/584,565, filed Jan. 9, 2012, all of which applications are hereby incorporated by reference in their entireties herein.

### BACKGROUND OF THE INVENTION

Influenza A virus causes one of the most widespread infections in humans. Between 10% and 20% of the U.S. population suffer from seasonal influenza each year. While most individuals recover in one to two weeks, the very young, the elderly and persons with chronic medical conditions can develop post-flu pneumonia and other lethal complications. The causative agent of influenza is influenza virus, a myxovirus that readily develops new strains through a process of reassortment and mutation of the segmented viral genome. Highly virulent strains of type A influenza virus can produce epidemics and pandemics. The emergence and global spread of the 2009 pandemic H1N1 influenza virus demonstrates that there is a need in the field for strategies to control influenza infection.

Vaccines are the best option for the prophylaxis and control of a pandemic. However, the lag time between virus identification and vaccine distribution exceeds six months and concerns regarding vaccine safety are a growing issue leading to vaccination refusal. In the short term, antiviral therapy is vital to control the spread of influenza. However, there currently exist only a few approved anti-influenza drugs, including Tamiflu and Relenza. Tamiflu directly targets the viral neuraminidase enzyme, thereby resulting in rapid development of drug resistance. Although Relenza targets the same enzyme, resistance to seasonal H1N1 is not as predominant as with Tamiflu. It is expected that a product that boosts the host's defenses would be least prone to resistance development.

The spreading of influenza type A virus drug resistance and the emergence of pandemic strains such as the novel H1N1 strains have made urgent the discovery of novel therapeutic targets for influenza virus. Small molecule antiviral therapies are critical as a first line defense against new threats. Influenza virus infection activates a variety of host signaling pathways, some of which are required for the host antiviral response, but others that are exploited by the virus for its replication and propagation.

Because of the threat posed by influenza virus both to public health and as a potential agent of bioterrorism, developing therapeutics to control seasonal influenza and the increasing threat of pandemic influenza is one of this nation's highest priorities. Transmission of H1N1 influenza virus from the swine to human shows great urgency for the development of an effective agent against influenza viruses. Infectious outbreaks with influenza virus are associated with high disease-related mortality and significant socioeconomic impact. Influenza causes an acute febrile illness and results in variable degrees of systemic symptoms, ranging from mild fatigue to respiratory failure and death. These symptoms contribute to significant loss of workdays, human suffering, mortality, and significant morbidity. In the U.S., annual epidemics cause approximately 300,000 hospitalizations and 36,000 deaths. In addition, three influenza pandemics (1918, 1957 and 1968) during the recent century have together taken an enormous toll of millions of lives. The appearances of the avian H5N1 influenza virus in 2003, and the more recent pandemic H1N1 outbreak in 2009, serve as stark reminders that preparedness to meet the threat of new and infectious influenza virus is essential. Shortages in vaccines production and the time required to deliver a vaccine against a novel influenza virus strain present considerable challenges to public protection against influenza virus. To cover this gap in vaccine production, and to treat individuals already infected, stockpiling of antiviral drugs is becoming commonplace. Also the possibility of influenza virus as a bioterrorism agent and the spreading of influenza type A virus resistance to existing drugs have made the discovery of novel therapeutic targets for influenza virus urgent.

Infection with influenza virus results in the activation of a variety of intracellular signaling pathways (Kumar et al., 2008, J. Virol. 82:9880-9889; Konig 2010, Nature, 463:813-817; Shapira, 2009, Cell 139:1255-1267; Karlas, 2010, Nature 463:818-822) that are in part required to mount an antiviral response to infection, but also may be exploited by the virus to support its replication and propagation. One such pathway is mediated by the Class I phosphatidylinositol 3-Kinase (PI3K) (Ehrhardt, 2006, Cell Microbiol. 8:1336-1348; Shin et al., 2007, J. Gen. Virol. 88:942-950; Hale et al., 2006, Proc. Natl Acad. Sci. USA 103:14194-14199). PI3Ks represent a family of enzymes and structurally closely lipid kinases that catalyze the ATP-dependent phosphorylation of phosphoinositide substrates. The primary function of 3-phosphorylated inositol lipids is to mediate membrane recruitment of selected proteins, thereby mediating vesicle trafficking, cytoskeletal reorganization and signal transduction (Vanhaesebroeck, 2001, Annu. Rev. Biochem. 70:535-602). PI3K is a dimeric enzyme that is classified as I, II, or III, depending on their domain organization, i.e. subunit structure, regulation, and substrate selectivity. Class IA PI3K consists of regulatory (p85) and enzymatic (p110) subunits, existing in three isoforms (p110α, p110β and p110δ), whereas the class IB PI3K have only one member p110γ (enzymatic subunit) that associates with different regulatory subunits (p101, p84 and p87) (Vanhaesebroeck, 1997, Proc. Natl Acad. Sci. USA 94:4330-4335; Cantry, 1997, J. Biol. Chem. 272:19236-19241). The catalytic isoform p110δ of class IA PI3K is preferentially expressed in hematopoietic cells and plays an important role in CD4+ T cells, B cells, Natural Killer and regulatory T cells development and function (Clayton, 2002, J. Exp. Med. 196:753-763; Okkenhaug, 2002, Science 297:1031-1034; Okkenhaug et al., 2003, Nat. Rev. Immunol 3:317-330). The p110δ subunit is expressed predominantly by hematopoietic cells and plays an important role in B and T cell development and function. Although several in vitro studies have suggested that targeting of PI3Ks would be a useful antiviral strategy (Ehrhardt, 2006, Cell Microbiol. 8:1336-1348; Hale et al., 2006, Proc. Natl Acad. Sci. USA 103:14194-14199; Shin et al., 2007, J. Gen. Virol. 88:942-950), the broad blockade of PI3Ks would not be beneficial.

There is a need to identify novel methods of treating or preventing seasonal or pandemic influenza infection and its effects on the health of humans and animals. The presently available vaccines against influenza virus are not sufficiently versatile to provide protection against new mutant strains. Current drugs that block influenza virus replication do not eliminate the morbidity symptoms associated with virus infection: fever, malaise, weight loss. Further, due to the increasing rate of virus resistance to the presently available drugs and inherent toxicity of these drugs, there is a need in the art for novel drugs that overcome these limitations. The present invention satisfies this need.

### BRIEF SUMMARY OF THE INVENTION

The invention includes a composition comprising a compound of Formula (I), or a salt thereof:

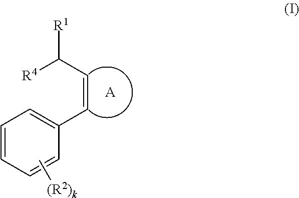

wherein in (I):
R¹ is selected from the group consisting of:

6-amine-9H-purin-9-yl, (9H-purin-6-yl)amino,

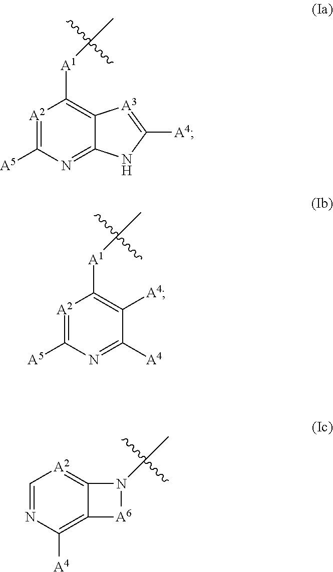

wherein in (Ia), (Ib) and (Ic):
A¹ is N(R⁸), O or S;
A² and A³ are independently C(R⁸) or N;
each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘOC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;
ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not:

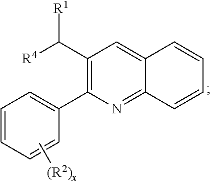

each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, (L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;

R⁴ is H, —C₁-C₆ alkyl, —C₁-C₆ fluoroalkyl or —C₁-C₆ heteroalkyl;

each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;

R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;

L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃ alkylene)ₘ-NH—(C₁-C₃ alkylene)ₘ-;

x is 0, 1, 2 or 3; and,

each occurrence of m is independently 0, 1 or 2.

In one embodiment, ring A is selected from the group consisting of pyridine, pyrimidine, quinoline, isoquinoline, 1,8-naphthyridine, 1,7-naphthyridine, 1,6-naphthyridine, 1,5-naphthyridine, pyrido[2,3-c]-pyridazine, pyrido[2,3-d]-pyrimidine, pyrido[2,3-b]-pyrazine, and 1,2,3,4-tetrahydro-1,8-naphthyridine. In another embodiment, R⁴ is H or C₁-C₆ alkyl. In yet another embodiment, R⁴ is H. In yet another embodiment, the compound of Formula (I) is selected from the group consisting of:

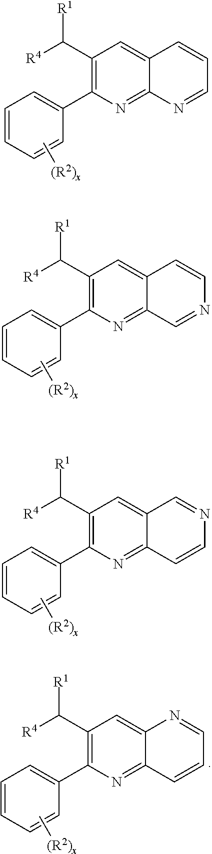

In yet another embodiment, the compound of Formula (I) is a compound of Formula (II) or a salt thereof:

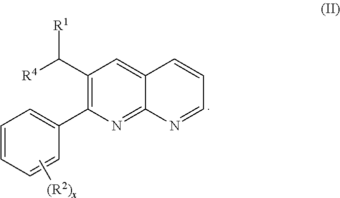

In yet another embodiment, the compound of Formula (II) is a compound of Formula (III) or a salt thereof:

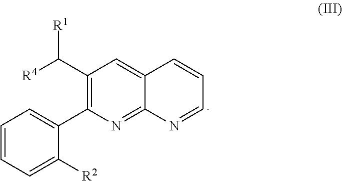

In yet another embodiment, the compound of Formula (I) is a compound of Formula (IV) or a salt thereof:

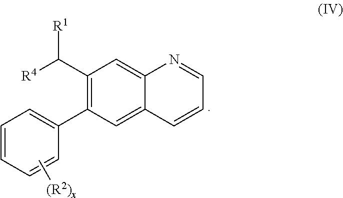

In yet another embodiment, the compound of Formula (I) is selected from the group consisting of: 9-((6-(2-chlorophenyl)quinolin-7-yl)methyl)-9H-purin-6-amine; 9-((6-(2-methylphenyl)quinolin-7-yl)methyl)-9H-purin-6-amine; 9-((2-(2-chlorophenyl)-1,8-naphthyridin-3-yl)methyl)-9H-purin-6-amine; 9-((2-(2-methylphenyl)-1,8-naphthyridin-3-yl)methyl)-9H-purin-6-amine; N-(1-(2-phenyl-1,8-naphthyridin-3-yl)propyl)-9H-purin-6-amine; a salt thereof; and any combinations thereof. In yet another embodiment, the composition further comprises at least one anti-influenza drug. In yet another embodiment, the at least one anti-influenza drug is selected from the group consisting of influenza combination drugs, entry and fusion inhibitors, integrase inhibitors, non-nucleoside reverse transcriptase inhibitors, nucleoside reverse transcriptase inhibitors, protease inhibitors, and combinations thereof. In yet another embodiment, the composition further comprises at least one immunomodulator.

The invention also includes a method of inhibiting replication of a virus in a cell. The method comprises contacting the cell with a composition comprising an inhibitor of PI3K p110 delta, wherein the contacting inhibits PI3K110 delta in the cell, thereby inhibiting replication of the virus in the cell, wherein the inhibitor is a compound of Formula (I) or a salt thereof:

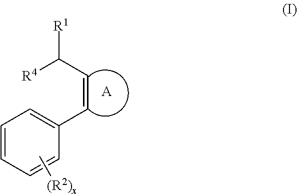

wherein in (I):
R¹ is selected from the group consisting of:

6-amine-9H-purin-9-yl, (9H-purin-6-yl)amino,

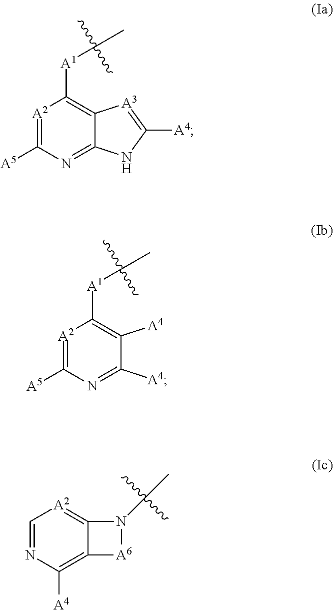

wherein in (Ia), (Ib) and (Ic):
A¹ is N(R⁸), O or S;
A² and A³ are independently C(R⁸) or N;
each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;

ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not:

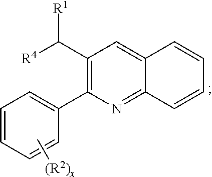

each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, (L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;

R⁴ is H, —C₁-C₆ alkyl, —C₁-C₆ fluoroalkyl or —C₁-C₆ heteroalkyl;

each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;

R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;

L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃ alkylene)ₘ-NH—(C₁-C₃ alkylene)ₘ-;

x is 0, 1, 2 or 3; and,

each occurrence of m is independently 0, 1 or 2.

The invention also includes a method of inhibiting pathogenesis of a virus in a mammalian cell. The method comprises contacting the cell with a pharmaceutically acceptable composition comprising a therapeutically effective amount of an inhibitor of PI3K p110 delta, wherein the contacting inhibits PI3K p110 delta in the cell, thereby inhibiting pathogenesis of the virus in the mammalian cell, wherein the inhibitor is a compound of formula (I) or a salt thereof:

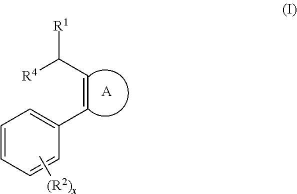

wherein in (I):
R¹ is selected from the group consisting of:

6-amine-9H-purin-9-yl, (9H-purin-6-yl)amino,

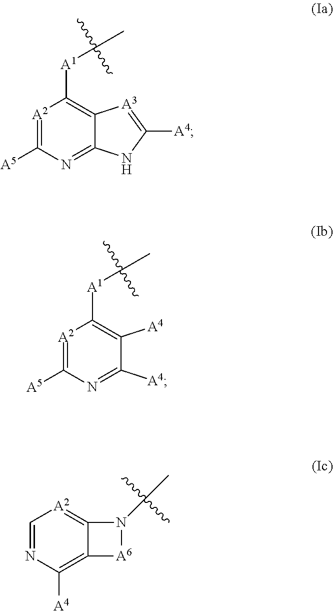

wherein in (Ia), (Ib) and (Ic):
A¹ is N(R⁸), O or S;
A² and A³ are independently C(R⁸) or N;
each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;

ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not:

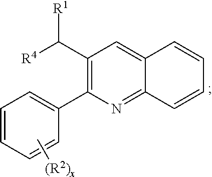

each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, (L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R, -(L)ₘ-CH(R⁸)₂, -(L)ₘ-N(R⁸)₂, -(L)ₘ-C(═O)N(R⁸)₂, -(L)ₘOC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;

R⁴ is H, —C₁-C₆ alkyl, —C₁-C₆ fluoroalkyl or —C₁-C₆ heteroalkyl;

each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;

R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;

L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃ alkylene)ₘ-NH—(C₁-C₃ alkylene)ₘ-;

x is 0, 1, 2 or 3; and,

each occurrence of m is independently 0, 1 or 2.

The invention also includes a method of treating or preventing infection by a virus in a mammal in need thereof. The method comprises administering a pharmaceutically acceptable composition comprising a therapeutically effective amount of an inhibitor of phosphoinositide 3 kinase (PI3K) isoform p110 delta to the mammal, wherein the inhibitor interferes with PI3K p110 delta activation and replication of the virus in the mammal, thereby treating or preventing the infection in the mammal, wherein the inhibitor is a compound of formula (I) or a salt thereof:

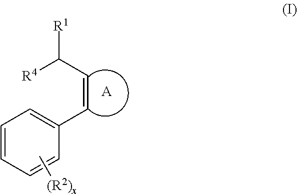

wherein in (I):
R¹ is selected from the group consisting of:

6-amine-9H-purin-9-yl, (9H-purin-6-yl)amino,

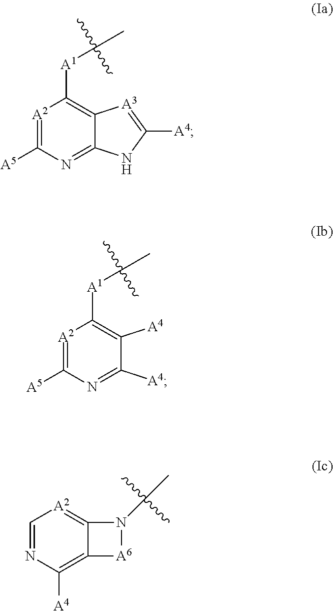

wherein in (Ia), (Ib) and (Ic):
A¹ is N(R⁸), O or S;
A² and A³ are independently C(R⁸) or N; each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;

ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not:

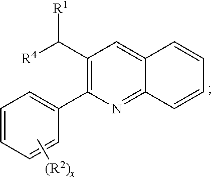

each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, (L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘOC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;

R⁴ is H, —C₁-C₆ alkyl, —C₁-C₆ fluoroalkyl or —C₁-C₆ heteroalkyl;

each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;

R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;

L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃ alkylene)ₘ-NH—(C₁-C₃ alkylene)ₘ-;

x is 0, 1, 2 or 3; and,

each occurrence of m is independently 0, 1 or 2.

In one embodiment, the virus is influenza. In another embodiment, ring A is selected from the group consisting of pyridine, pyrimidine, quinoline, isoquinoline, 1,8-naphthyridine, 1,7-naphthyridine, 1,6-naphthyridine, 1,5-naphthyridine, pyrido[2,3-c]-pyridazine, pyrido[2,3-d]-pyrimidine, pyrido[2,3-b]-pyrazine, and 1,2,3,4-tetrahydro-1,8-naphthyridine. In yet another embodiment, R⁴ is H or C₁-C₆ alkyl. In yet another embodiment, R⁴ is H. In yet another embodiment, the compound of Formula (I) is selected from the group consisting of:

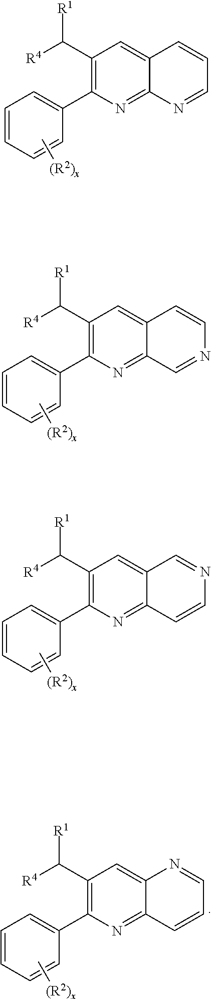

In yet another embodiment, the compound of Formula (I) is a compound of Formula (II) or a salt thereof:

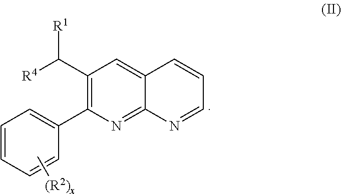

In yet another embodiment, the compound of Formula (II) is a compound of Formula (III) or a salt thereof:

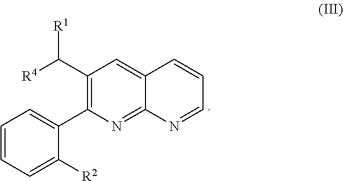

In yet another embodiment, the compound of Formula (I) is a compound of Formula (IV) or a salt thereof:

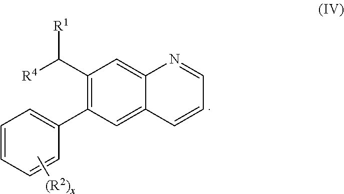

In yet another embodiment, the compound of Formula (I) is selected from the group consisting of: 9-((6-(2-chlorophenyl)quinolin-7-yl)methyl)-9H-purin-6-amine; 9-((6-(2-methylphenyl)quinolin-7-yl)methyl)-9H-purin-6-amine; 9-((2-(2-chlorophenyl)-1,8-naphthyridin-3-yl)methyl)-9H-purin-6-amine; 9-((2-(2-methylphenyl)-1,8-naphthyridin-3-yl)methyl)-9H-purin-6-amine; N-(1-(2-phenyl-1,8-naphthyridin-3-yl)propyl)-9H-purin-6-amine; a salt thereof; and any combinations thereof. In yet another embodiment, the composition further comprises at least one anti-influenza drug. In yet another embodiment, the at least one anti-influenza drug is selected from the group consisting of influenza combination drugs, entry and fusion inhibitors, integrase inhibitors, non-nucleoside reverse transcriptase inhibitors, nucleoside reverse transcriptase inhibitors, protease inhibitors, and combinations thereof. In yet another embodiment, the composition further comprises at least one immunomodulator. In yet another embodiment, the cell is human. In yet another embodiment, the mammal is human. In yet another embodiment, the inhibitor interferes with pathogenesis of the virus.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1a-1c, is a series of images illustrating that PI3K p110 delta is expressed in lung epithelial cells and that PI3K p110 delta is required for influenza virus replication.

FIG. 2, comprising FIG. 2a-2b, is a series of images illustrating that inhibition of PI3K p110 delta protects mice from lethal influenza virus infection.

FIG. 3 is a graph demonstrating reduced influenza virus titers in p110δ−/− mice. Lung influenza virus titers are reduced in p110δ−/− mice on day 6 and 10 post infection. C57B1/6 (n=6-13) and p110δ−/− mice (n=6-9) were infected intranasally with 3 TCID₅₀ of influenza virus strain PR8 and lung viral load was determined by real-time PCR, using as standard curve dilutions of cDNA synthesized from an influenza virus stock of known concentration. Each symbol represents one animal and the horizontal line represents the mean viral load. Data were pooled from 3 independent experiments shown.

FIG. 4 illustrates the structural variation of the PI3K inhibitor pharmacophore.

FIG. 5 is a scheme illustrating synthesis of the compounds of the invention 7a and 7b; a=propionyl chloride, Et₃N, 1,4-dioxane, rt, overnight; b=Cs₂CO₃, DMF, 65° C., overnight (81%, 2 steps); b═NBS, benzoyl peroxide, CCl₄, 88° C., 4 h (80%); d=POCl₃, 80° C., 3 h (74%); e=adenine, Cs₂CO₃, DMF, 80° C., 3 h (83%); f=Pd(PPh₃)₄, Na₂CO₃, MeCN, H₂O (3:1), 100° C., 3 h (93%).

FIG. 6 is a graph illustrating the inhibition of influenza virus (plaques/mL vs. concentration) by compounds 7a, 7b (from FIG. 5), and IC87114 vs. control.

FIG. 7 is an image illustrating the hinge interaction of target compound with the kinase domain of p110δ. The adenine ring has two interactions with the main chain atoms.

FIG. 8 is an image illustrating the chemical structure of CAL-101 and a compound of the invention.

FIG. 9 is an image illustrating the hinge interaction of IC87114 with the kinase domain of p110δ. The adenine ring has two interactions with main chain atoms of the hinge.

FIG. 10 is a scheme illustrating the synthesis of compounds of the derivatives.

### DETAILED DESCRIPTION OF THE INVENTION

The invention includes compositions and methods for regulating PI3K p110 delta kinase in a cell thereby providing a means for reducing or inhibiting virus infection or replication in the cell.

In one embodiment, the invention includes an inhibitor of the PI3K p110 delta kinase. In another embodiment, the inhibitor is a small molecule.

In one embodiment, the virus is influenza. In another embodiment, the inhibitor interferes with influenza virus pathogenesis. In yet another embodiment, the virus is a retrovirus. In yet another embodiment, the retrovirus is HIV. In yet another embodiment, the inhibitor interferes with retroviral pathogenesis.

The invention further includes a method of treating or preventing virus infection in in a cell or mammal. The method comprises administering to a cell or mammal an effective amount of a composition comprising an inhibitor of the invention. The administering of the composition of the invention to the cell or mammal interferes with PI3K p110 delta activation and replication of the virus in the cell or mammal, thereby treating or preventing the virus infection.

### DEFINITIONS

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of 20% or ±10%, more preferably ±5%, even more preferably ±1%, and still more preferably ±0.1% from the specified value, as such variations are appropriate to perform the disclosed methods.

A “subject” or “patient,” as used therein, may be a human or non-human mammal. Non-human mammals include, for example, livestock and pets, such as ovine, bovine, porcine, canine, feline and murine mammals. Preferably, the subject is human.

The term “virus” as used herein is defined as a particle consisting of nucleic acid (RNA or DNA) enclosed in a protein coat, with or without an outer lipid envelope, which is capable of replicating within a whole cell.

As used herein, the term “endogenous” refers to any material from or produced inside an organism, cell, tissue or system. As used herein, the term “exogenous” refers to any material introduced from or produced outside an organism, cell, tissue or system.

As used herein, the term “modulate” is meant to refer to any change in biological state, i.e. increasing, decreasing, and the like. For example, the term “modulate” refers to the ability to regulate positively or negatively the expression, stability or activity of p110 delta, including but not limited to transcription of PI3K p110 delta mRNA, stability of PI3K p110 delta mRNA, translation of PI3K p110 delta mRNA, stability of PI3K p110 delta polypeptide, PI3K p110 delta post-translational modifications, PI3K p110 delta activity, or any combination thereof. Further, the term modulate may be used to refer to an increase, decrease, masking, altering, overriding or restoring of activity, including but not limited to, PI3K p110 delta activity.

As used herein, the term “inhibit” is meant to refer to a decrease change in biological state. For example, the term “inhibit” refers to the ability to regulate negatively the expression, stability or activity of p110 delta, including but not limited to transcription of PI3K p110 delta mRNA, stability of PI3K p110 delta mRNA, translation of PI3K p110 delta mRNA, stability of PI3K p110 delta polypeptide, PI3K p110 delta post-translational modifications, PI3K p110 delta activity, PI3K p110 delta signaling pathway or any combination thereof.

As used herein, the term “an inhibitor of p110 delta,” “an inhibitor of PI3K delta” or “an inhibitor of PI3Kδ” refers to any compound or molecule that detectably inhibits p110 delta.

A “PI3K p110 delta antagonist” is a composition of matter which, when administered to a mammal such as a human, detectably inhibits a biological activity attributable to the level or presence of p110 delta.

“Parenteral” administration of an immunogenic composition includes, for example, subcutaneous (s.c.), intravenous (i.v.), intramuscular (i.m.), or intrasternal injection, or infusion techniques.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “composition,” “pharmaceutical composition” or “pharmaceutically acceptable composition” refers to a mixture of at least one compound or molecule useful within the invention with a pharmaceutically acceptable carrier. The pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The term “therapeutic” as used herein means a treatment and/or prophylaxis. A therapeutic effect is obtained by suppression, remission, or eradication of a disease state.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder.

As another example, administration of the agent after clinical manifestation of the disease to combat the symptoms of the disease comprises “treatment” of the disease. This includes for instance, prevention of viral infection or replication.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection or replication as determined by any means suitable in the art.

As used herein, an “instructional material” includes a publication, a recording, a diagram, or any other medium of expression that may be used to communicate the usefulness of the compositions and methods of the invention. The instructional material of the kit of the invention may, for example, be affixed to a container that contains the nucleic acid, peptide, and/or composition useful with the invention or be shipped together with a container that contains the nucleic acid, peptide, and/or composition. Alternatively, the instructional material may be shipped separately from the container with the intention that the instructional material and the compound be used cooperatively by the recipient.

As used herein, the term “6-amine-9H-purin-9-yl” refers to

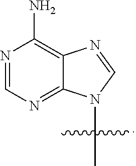 [SMILES: *n1cnc2c(N)ncnc21]

As used herein, the term “(9H-purin-6-yl)amino” refers to

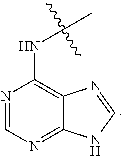 [SMILES: *Nc1ncnc2[nH]cnc12]

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C—N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

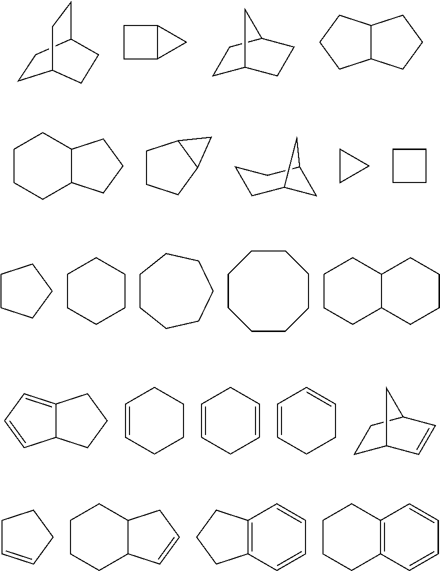

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, Sand N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

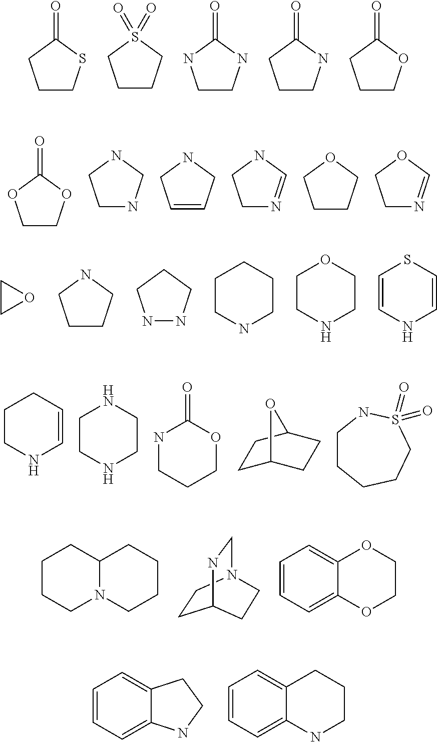

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized 7Z (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heteroaryl” or “heteroaromatic” refers to a heterocycle having aromatic character. A polycyclic heteroaryl may include one or more rings that are partially saturated. Examples include the following moieties:

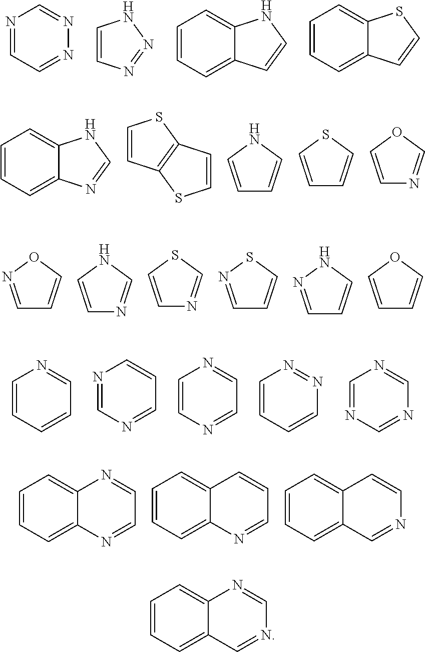

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

### Description

The present invention relates to the discovery of a series of novel selective PI3K p110delta inhibitors. These inhibitors were identified using a human active site model and ligand docking screening methods of the invention.

In one embodiment, the inhibitors exhibit selectivity for the delta isoform of PI3K p110 and exhibit antiviral activity.

In one embodiment, the inhibitors exhibit anti-influenza activity. In another embodiment, the inhibitors of the invention exhibit broad reactivity against more than one strain and subtypes of influenza. In yet another embodiment, the inhibitors exhibit broad reactivity against all strains and subtypes of influenza, irrespective of mutations or gene re-assortments of the surface proteins that may occur.

In one embodiment, the inhibitors are useful for therapies against seasonal and pandemic influenza virus strains, as well as other viruses. The inhibitors may be used alone or in combination with other anti-viral agents and/or anti-inflammatory agents.

### Compounds

The compounds of the invention may be synthesized using techniques well-known in the art of organic synthesis.

In one aspect, the invention includes a compound of Formula (I), or a salt thereof:

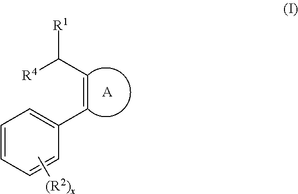

wherein in (I):
R¹ is selected from the group consisting of:

6-amine-9H-purin-9-yl,

(9H-purin-6-yl)amino,

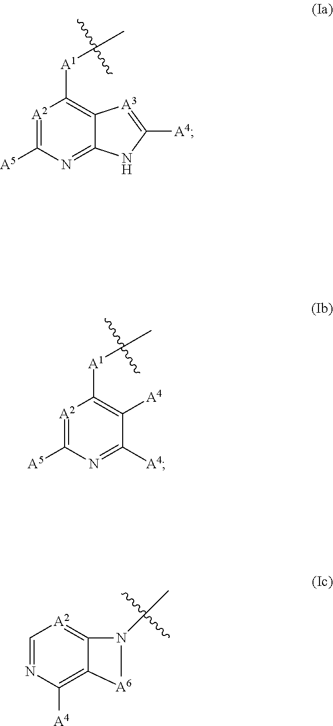

wherein in (Ia), (Ib) and (Ic):
A¹ is N(R⁸), O or S;
A² and A³ are independently C(R⁸) or N; each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;

ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not:

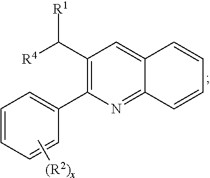

each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, (L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;

R⁴ is H, —C₁-C₆ alkyl, —C₁-C₆ fluoroalkyl or —C₁-C₆ heteroalkyl;

each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃—Co heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;

R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;

L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃ alkylene)ₘ-NH—(C₁-C₃ alkylene)ₘ-;

x is 0, 1, 2 or 3; and,

each occurrence of m is independently 0, 1 or 2.

In one embodiment, ring A is a monocyclic aryl ring, optionally substituted with 0-3 substituents selected from R³. In another embodiment, ring A is a bicyclic aryl ring, optionally substituted with 0-3 substituents selected from R³. In yet another embodiment, ring A is a monocyclic heteroaryl ring, wherein the aryl or heteroaryl ring is optionally substituted with 0-3 substituents selected from R³. In yet another embodiment, ring A is a bicyclic heteroaryl ring, optionally substituted with 0-3 substituents selected from R³. In yet another embodiment, ring A is selected from the group consisting of pyridine, pyrimidine, quinoline, isoquinoline, 1,8-naphthyridine, 1,7-naphthyridine, 1,6-naphthyridine, 1,5-naphthyridine, pyrido[2,3-c]-pyridazine, pyrido[2,3-d]-pyrimidine, pyrido[2,3-b]-pyrazine, and 1,2,3,4-tetrahydro-1,8-naphthyridine. In yet another embodiment, R⁴ is H or C₁-C₆ alkyl. In yet another embodiment, R⁴ is H.

In one embodiment, the compound of Formula (I) is selected from the group consisting of:

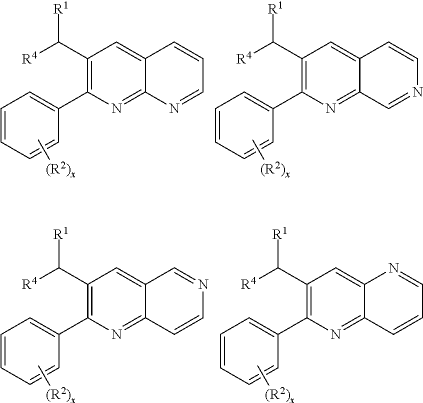

In one embodiment, the compound of Formula (I) is a compound of Formula (II) or a salt thereof:

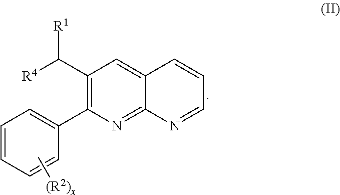

In one embodiment, the compound of Formula (I) is a compound of Formula (III) or a salt thereof:

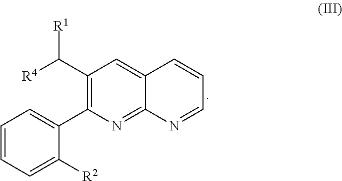

In one embodiment, the compound of Formula (I) is a compound of Formula (IV) or a salt thereof:

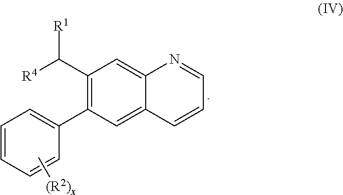

In one embodiment, the compound of the invention is selected from the group consisting of:
9-((6-(2-chlorophenyl)quinolin-7-yl)methyl)-9H-purin-6-amine;
9-((6-(2-methylphenyl)quinolin-7-yl)methyl)-9H-purin-6-amine;
9-((2-(2-chlorophenyl)-1,8-naphthyridin-3-yl)methyl)-9H-purin-6-amine;
9-((2-(2-methylphenyl)-1,8-naphthyridin-3-yl)methyl)-9H-purin-6-amine;
N-(1-(2-phenyl-1,8-naphthyridin-3-yl)propyl)-9H-purin-6-amine;
   combinations thereof, and a salt thereof.

### Synthesis

Compounds of formula (I) may be prepared by synthetic routes known to those skilled in the art.

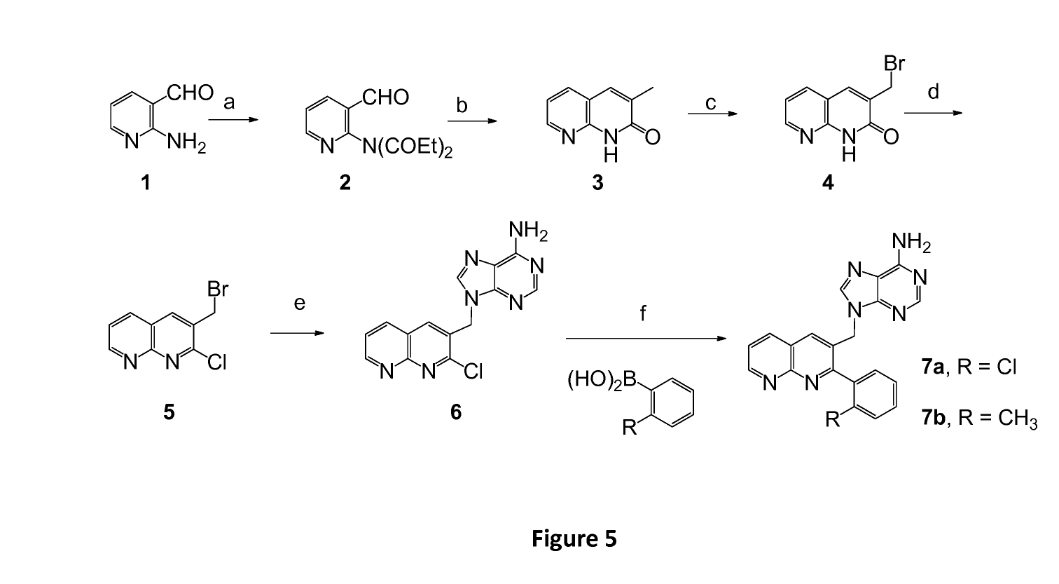

Selected examples of a compound of Formula (I) may be prepared according to the synthetic scheme outlined in FIG. 5. Compound 1 may be diacylated using an acyl chloride, yielding compound 2. Cyclization of compound 2 under basic condition yields compound 3, which may then be halogenated under free radical condition to yield compound 4. Halogenation of compound 4 yields compound 5, which may then be coupled with an amine to yield compound 6. Suzuki coupling may be used to derivatize compound 6, yielding compound 7a or 7b.

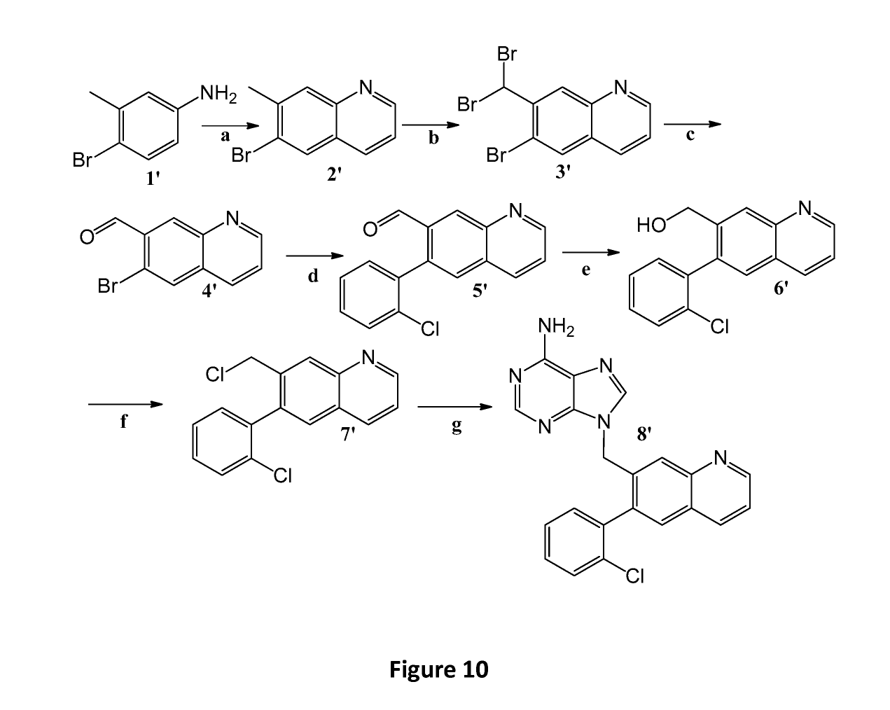

Selected examples of a compound of Formula (I) may be prepared according to the synthetic scheme outlined in FIG. 10. In FIG. 10, compound 1′ may be condensed with glycerol under acidic conditions to yield compound 2′. Compound 2′ may be brominated to yield compound 3′, which may be hydrolyzed to compound 4′. Compound 4′ may be derivatized via a Suzuki coupling reaction, yielding compound 5′. Reduction, followed by chlorination, yields compound 7′, which be coupled with a nucleophile under optionally basic conditions to yield compound 8′. Representative procedures for selected steps in this synthetic scheme may be found in Patent Application Publications No. US 2007/0265272, WO 2007/075424, WO 2008/118454 and WO 2008/118468.

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tethrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a pro drug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4th Ed., Vols. A and B (Plenum 2000,2001), and Green and Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

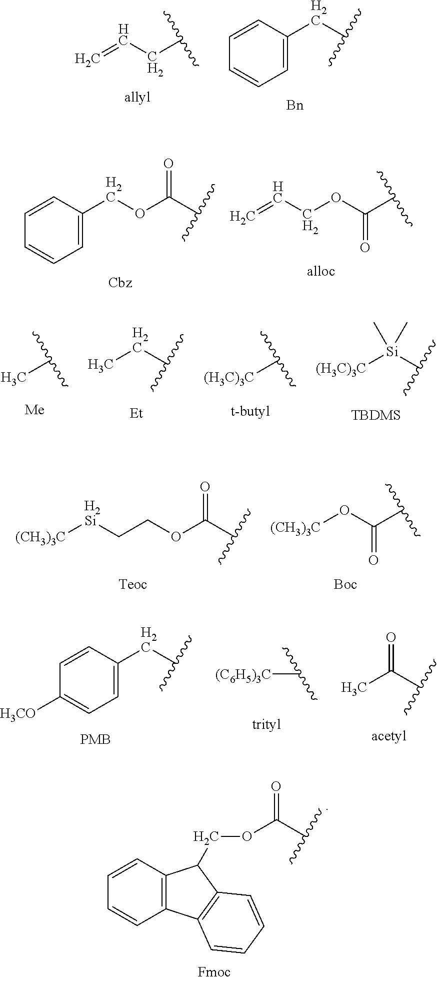

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Salts

The compounds useful within the invention may form salts with acids or bases, and such salts are included in the present invention. In one embodiment, the salts are pharmaceutically-acceptable salts. The term “salts” embraces addition salts of free acids or free bases that are compounds useful within the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds useful within the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding compound by reacting, for example, the appropriate acid or base with the compound.

### Methods

In one aspect, the invention includes a method of inhibiting virus replication. The method comprises the step of inhibiting phosphoinositide 3 kinase (PI3K) isoform p110 delta in a cell. The step comprises contacting the cell with a pharmaceutically acceptable composition comprising an inhibitor of PI3K p110 delta. In one embodiment, the inhibitor of PI3K p110 delta is a small molecule compound. In one embodiment, the virus is influenza. In another embodiment, the virus is a retrovirus, preferably HIV.

In one embodiment, the small molecule compound includes a compound of Formula (I), or a pharmaceutically acceptable salt thereof:

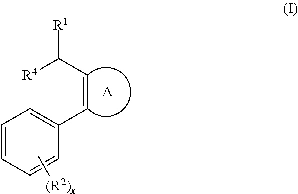

wherein in (I):
R¹ is selected from the group consisting of:

6-amine-9H-purin-9-yl,

(9H-purin-6-yl)amino,

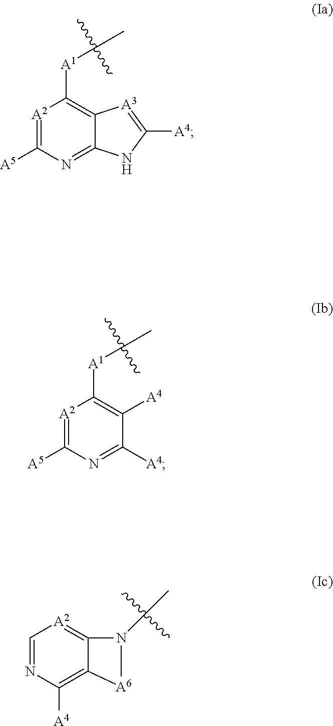

wherein in (Ia), (Ib) and (Ic):
A¹ is N(R⁸), O or S;
A² and A³ are independently C(R⁸) or N; each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;

ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not:

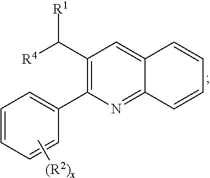

each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, (L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;

R⁴ is H, —C₁-C₆ alkyl, —C₁-C₆ fluoroalkyl or —C₁-C₆ heteroalkyl;

each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;

R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;

L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃ alkylene)ₘ-NH—(C₁-C₃ alkylene)ₘ-;

x is 0, 1, 2 or 3; and,

each occurrence of m is independently 0, 1 or 2.

In one embodiment, ring A is a monocyclic aryl ring, optionally substituted with 0-3 substituents selected from R³. In another embodiment, ring A is a bicyclic aryl ring, optionally substituted with 0-3 substituents selected from R³. In yet another embodiment, ring A is a monocyclic heteroaryl ring, wherein the aryl or heteroaryl ring is optionally substituted with 0-3 substituents selected from R³. In yet another embodiment, ring A is a bicyclic heteroaryl ring, optionally substituted with 0-3 substituents selected from R³. In yet another embodiment, ring A is selected from the group consisting of pyridine, pyrimidine, quinoline, isoquinoline, 1,8-naphthyridine, 1,7-naphthyridine, 1,6-naphthyridine, 1,5-naphthyridine, pyrido[2,3-c]-pyridazine, pyrido[2,3-d]-pyrimidine, pyrido[2,3-b]-pyrazine, and 1,2,3,4-tetrahydro-1,8-naphthyridine. In yet another embodiment, R⁴ is H or C₁-C₆ alkyl. In yet another embodiment, R⁴ is H.

In one embodiment, the compound of Formula (I) is selected from the group consisting of:

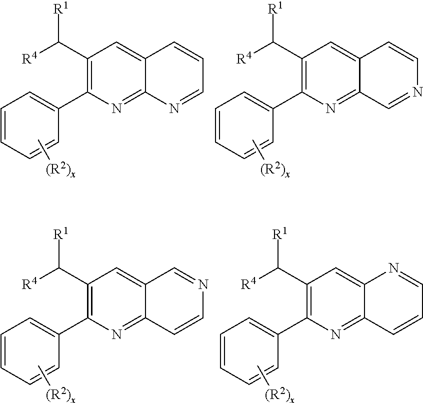

In one embodiment, the compound of Formula (I) is a compound of Formula (II) or a salt thereof:

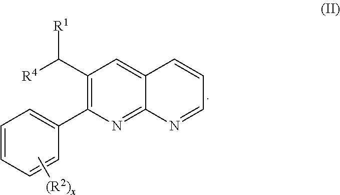

In one embodiment, the compound of Formula (I) is a compound of Formula (III) or a salt thereof:

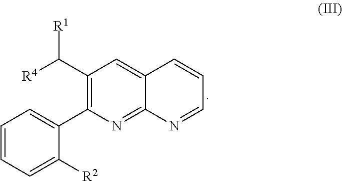

In one embodiment, the compound of Formula (I) is a compound of Formula (IV) or a salt thereof:

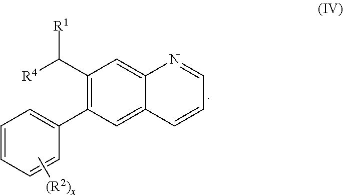

In one embodiment, the compound of the invention is selected from the group consisting of:
9-((6-(2-chlorophenyl)quinolin-7-yl)methyl)-9H-purin-6-amine;
9-((6-(2-methylphenyl)quinolin-7-yl)methyl)-9H-purin-6-amine;
9-((2-(2-chlorophenyl)-1,8-naphthyridin-3-yl)methyl)-9H-purin-6-amine;
9-((2-(2-methylphenyl)-1,8-naphthyridin-3-yl)methyl)-9H-purin-6-amine;
N-(1-(2-phenyl-1,8-naphthyridin-3-yl)propyl)-9H-purin-6-amine;
   combinations thereof, and a salt thereof.

In another aspect, the invention includes a method of inhibiting viral pathogenesis. The method comprises the step of inhibiting phosphoinositide 3 kinase (PI3K) isoform p110 delta in a cell. The step comprises contacting the cell with a pharmaceutically acceptable composition comprising an inhibitor of PI3K p110 delta. In one embodiment, the inhibitor of PI3K p110 delta is a small molecule compound. In another embodiment, the small molecule compound includes a compound of Formula (I), or a pharmaceutically acceptable salt thereof.

In yet another aspect, the invention includes a method of treating or preventing viral infection in a mammal. The method comprises the step of administering an effective amount of a composition comprising an inhibitor of phosphoinositide 3 kinase (PI3K) isoform p110 delta to the mammal in need thereof, wherein the inhibitor interferes with PI3K p110 delta activation and viral replication. In one embodiment, the inhibitor of PI3K p110 delta is a small molecule compound. In another embodiment, the small molecule compound includes a compound of Formula (I), or a pharmaceutically acceptable salt thereof.

In one embodiment, the composition further comprises at least one anti-influenza drug. In another embodiment, the at least one anti-influenza drug is selected from the group consisting of influenza combination drugs, entry and fusion inhibitors, integrase inhibitors, non-nucleoside reverse transcriptase inhibitors, nucleoside reverse transcriptase inhibitors, and protease inhibitors.

In one embodiment, the composition further comprises at least one anti-HIV drug. In another embodiment, the at least one anti-HIV drug is selected from the group consisting of HIV combination drugs, entry and fusion inhibitors, integrase inhibitors, non-nucleoside reverse transcriptase inhibitors, nucleoside reverse transcriptase inhibitors, and protease inhibitors.

In one embodiment, the composition further comprises at least one immunomodulator. In another embodiment, the at least one immunomodulator is an anti-inflammatory agent. In yet another embodiment, the anti-inflammatory agent is non-steroidal. In yet another embodiment, the anti-inflammatory agent is a non-steroidal anti-inflammatory (NSAID) agent.

PI3K p110 Delta Inhibitor

The invention includes the use of small molecule compounds to inhibit PI3K p110 delta, a component of the PI3K p110 delta signaling pathway, or any combination thereof. In a non-limiting example, IC87114, a selective inhibitor of PI3K p110 delta is useful in inhibiting PI3K p110 delta signaling pathway in a cell. The disclosure presented herein demonstrates that PI3K p110 delta inhibitors are able to inhibit PI3K p110 delta, a component of the PI3K p110 delta signaling pathway, or a combination thereof, to provide a therapeutic benefit in infected mammals. For example, the PI3K p110 delta inhibitor in the form of a small molecule compound may significantly reduce viral loads of infected mammals. In addition, the PI3K p110 delta inhibitor is able to reduce the number of cellular infiltration compared to a mammal not treated with the inhibitor. Also, the treatment with the inhibitor reduces the number of inflammatory cells infiltrating the cells of infected mammals. Thus, the inhibitor of the invention provides a means to regulate viral replication and pathogenesis. That is, any inhibitor of the invention that may therapeutically target PI3K p110 delta provides a therapy against viral infection. Thus, both genetic and pharmacologic means of PI3K p110 delta signaling inhibition is included in the invention as a useful strategy against viral infection.

### Combinational Therapy

In one aspect, the compositions of the invention relating to inhibiting p110 delta, a component of PI3K p110 delta signaling pathway, or any combinations thereof, may be combined with one or more immunomodulators. A preferred composition has an effective amount of a PI3K p110 delta inhibitor to inhibit or reduce viral infection in combination with an effective amount of one or more, anti-inflammatory agents, preferably non-steroidal anti-inflammatory agents to reduce inflammatory responses in the subject.

Immunomodulators include immune suppressors or enhancers and anti-inflammatory agents. Preferred immunomodulators are anti-inflammatory agents. The anti-inflammatory agent may be non-steroidal, steroidal, or a combination thereof.

Preferred anti-inflammatory agents are non-steroidal anti-inflammatory (NSAID) agents. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

In one embodiment, immunomodulators are COX-2 inhibitors such as celecoxib and aminosalicylate drugs such as mesalazine and sulfasalazine. In a preferred embodiment, the disclosed composition contains an effective amount of an inhibitor of PI3K p110 delta to inhibit or reduce viral infection in a subject in combination with an effective amount of celecoxib and mesalazine to reduce inflammatory responses in the subject.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, predisone, beclomethasone dipropionate, triamcinolone, and mixtures thereof.

In another aspect, the compounds useful within the methods of the invention may be used in combination with one or more additional compounds useful for treating HIV infections. These additional compounds may comprise compounds that are commercially available or synthetically accessible to those skilled in the art. These additional compounds are known to treat, prevent, or reduce the symptoms of HIV infections.

In non-limiting examples, the compounds useful within the invention may be used in combination with one or more of the following anti-HIV drugs:

HIV Combination Drugs: efavirenz, emtricitabine or tenofovir disoproxil fumarate (Atripla®/BMS, Gilead); lamivudine or zidovudine (Combivir®/GSK); abacavir or lamivudine (Epzicom®/GSK); abacavir, lamivudine or zidovudine (Trizivir®/GSK); emtricitabine, tenofovir disoproxil fumarate (Truvada®/Gilead).

Entry and Fusion Inhibitors: maraviroc (Celsentri®, Selzentry®/Pfizer); pentafuside or enfuvirtide (Fuzeon®/Roche, Trimeris).

Integrase Inhibitors: raltegravir or MK-0518 (Isentress®/Merck).

Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).

Nucleoside Reverse Transcriptase Inhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddI, dideoxyinosine or didanosine (Videx®/BMS); abacavir sulfate plus lamivudine (Epzicom®/GSK); stavudine, d4T, or estavudina (Zerit®/BMS); tenofovir, PMPA prodrug, or tenofovir disoproxil fumarate (Viread®/Gilead).

Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec); fosamprenavir (Telzir®, Lexiva®/GSK, Vertex); indinavir sulfate (Crixivan®/Merck); saquinavir mesylate (Invirase®/Roche); lopinavir or ritonavir (Kaletra®/Abbott); nelfinavir mesylate (Viracept®/Pfizer); ritonavir (Norvir®/Abbott).

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enz. Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

Routes of administration of any of the compositions of the invention include topical, oral, nasal, buccal, sublingual, rectal, pleural, peritoneal, vaginal, intramuscular, subcutaneous, transdermal, epidural, intrathecal or intravenous route.

The regimen of administration may affect what constitutes an effective amount. The therapeutic formulations may be administered to the subject either prior to or after the onset of a viral infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat a viral infection in the subject. An effective amount of the therapeutic compound necessary to achieve a therapeutic effect may vary according to factors such as the state of the disease or disorder in the subject; the age, sex, and weight of the subject; and the ability of the therapeutic compound to treat a viral infection in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a viral infection in a subject.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from individual to individual depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments 15 therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a second viral infection inhibitor) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments therebetween.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a viral infection in a subject.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

In a further embodiment, the present invention relates to a method for manufacturing a multi-layer tablet comprising a layer providing for the delayed release of one or more compounds of the invention, and a further layer providing for the immediate release of a medication for a viral infection. Using a wax/pH-sensitive polymer mix, a gastric insoluble composition may be obtained in which the active ingredient is entrapped, ensuring its delayed release.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, and polyvinylpyrrolidone. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a viral infection in a subject.

The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments therebetween after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments therebetween after drug administration.

### Dosing

The therapeutically effective amount or dose of a compound of the present invention will depend on the age, sex and weight of the subject, the current medical condition of the subject and the nature of the viral infection being treated. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the invention may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

In Vitro Validation of PI3K p110δ as an Antiviral Target

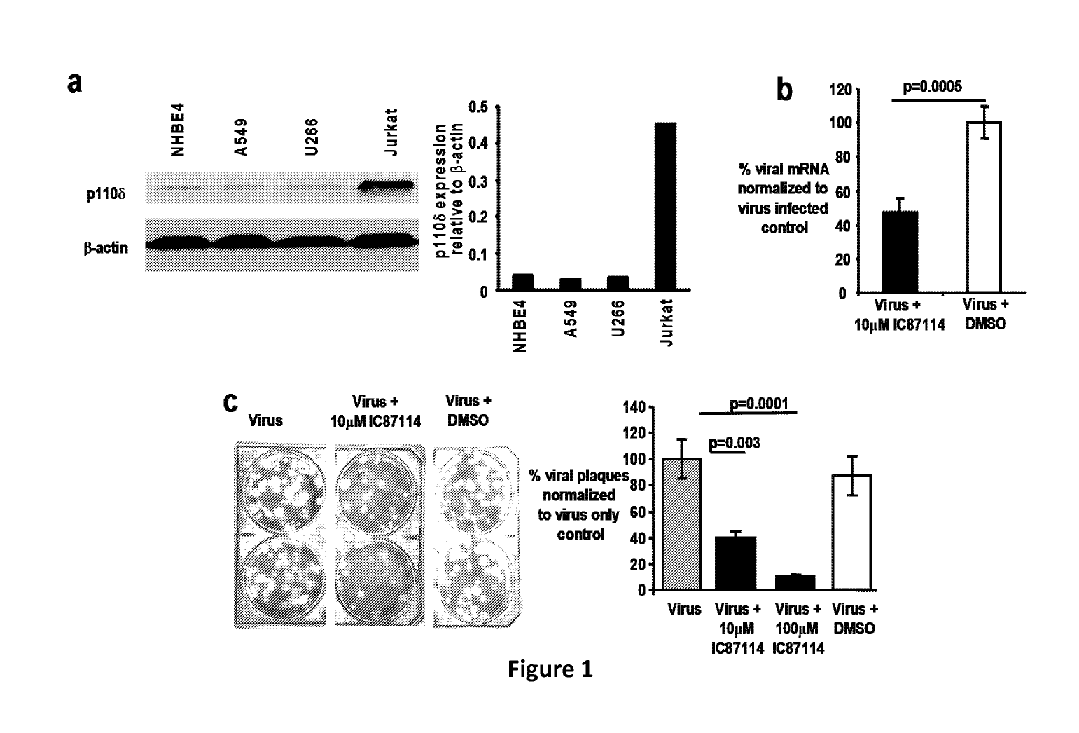

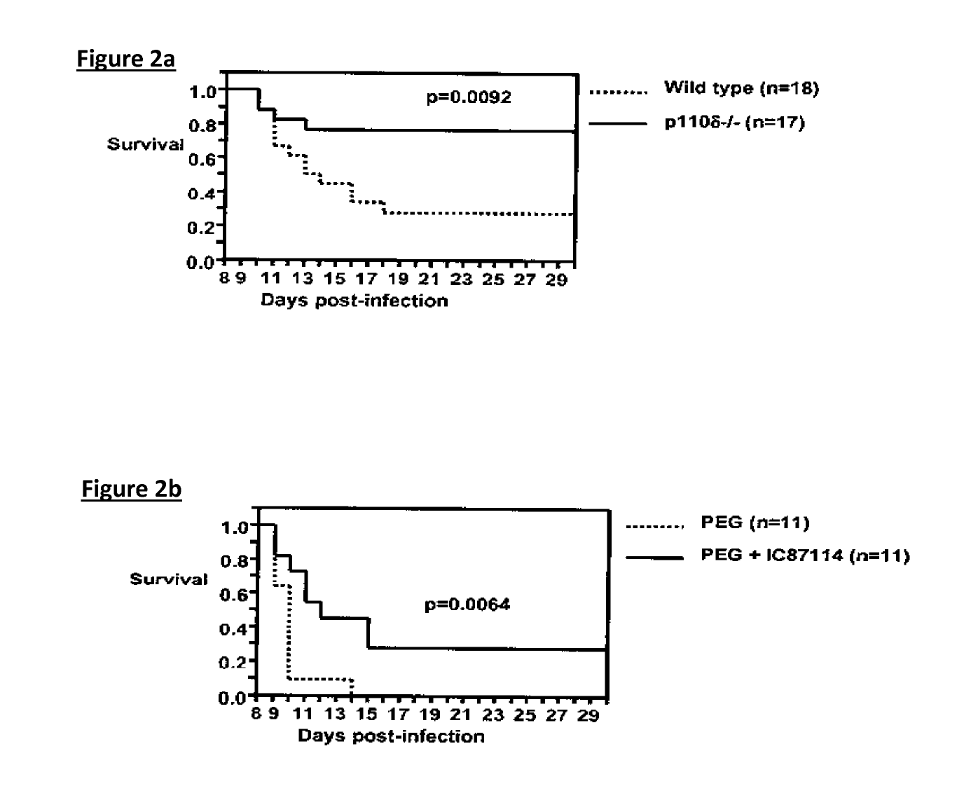

It has been demonstrated that p110δ isoform of phosphatidylinositol 3-kinase (PI3K) is expressed in both human lung epithelial A549 and normal primary human bronchial NHBE4 cells (FIG. 1A) by Western blotting. When PI3Kδ is inhibited in vitro by the selective inhibitor IC87114, viral replication in A549 lung epithelial cells is decreased as measured by mRNA expression and viral particle load. Validation that p110δ is an important target comes from in vivo studies that demonstrated that p110δ knockout mice were protected from a lethal challenge by a highly pathogenic in mice influenza virus strain (H7N7 A/Equine/London/1416/73) (FIG. 2A).

In Vivo Validation of PI3K p110δ as an Antiviral Target

It has been demonstrated that Influenza virus infected p110δ deficient mice exhibit reduced lung viral loads, inflammatory cytokines, morbidity, and mortality. When p110δ−/− (on C57B1/6 genetic background) and control C57B1/6 mice were infected i.n. with a sublethal dose (3 TCID₅₀) of influenza virus A/PR/8/34 (H1N1) and weight loss was monitored, it was observed that C57B1/6 control mice lost up to 25% of their initial body weight by day 9 post-infection, while p110δ−/− mice presented a maximum weight loss of only 5% of their initial body weight. p110δ−/− mice appeared healthier during influenza virus infection and displayed less labored breathing, compared to C57B1/6 controls. These findings demonstrate that p110δ signaling plays an important role in influenza virus associated morbidity.

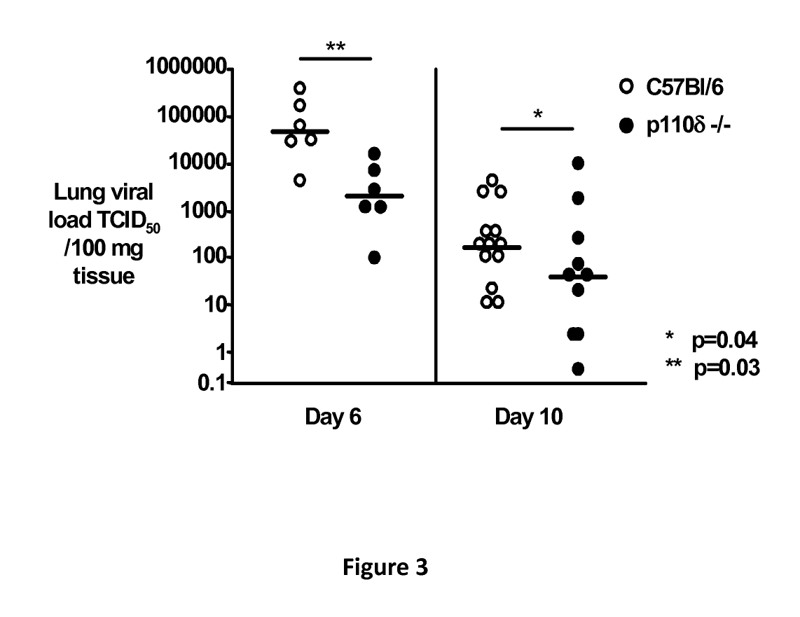

It was observed that the lung influenza viral titers in p110δ−/− mice were ˜10-fold lower than controls (FIG. 3). As discussed elsewhere herein, p110δ knockout mice were protected from lethal influenza challenge (FIG. 2A). In addition, pharmacological inhibition of p1106δ by IC87114 (50 mg/kg, po) also protected animals from lethal challenge with a virulent influenza virus strain (FIG. 2). Thus p110δ signaling plays an important role in influenza virus infection and pathogenesis, and serves as a novel target for therapies against seasonal and pandemic flu.

### Docking Studies

The design of new PI3K inhibitors began from the starting point of the selective p110δ inhibitor IC87114. The structure (Berndt, et al., 2010, Nature Chemical Biology 6:117-124) of murine p110δ bound to its selective ligand IC87114 (PI3Kδ IC₅₀=0.5 μM) (Sadhu, et al., 2003, J. Immunol. 170:2647-2654) has been published and was modified for use in the present drug design experiments. The structures docked into the enzyme were chosen from two alternative connections to adenine (A1 and A2), 35 2″,3″-substituted bicyclic rings (mostly 6, 6), 18 substituents on B, and 25 ortho substituents on C=Phenyl. GLIDE and QikProp generated parameters on 31,500 possible compounds, as well as the 13 x-ray ligands from PDB-2×38. The designed analogs maintain the H-bond donating/accepting pair of the adenine ring. The perpendicular aromatic rings with one ortho-substituent also appear to be critical for efficient binding. Interactions of these aromatic rings with hydrophobic residues Trp760, Ile777, Pro758 and Met752 are operative. A series of PI3Kδ inhibitors from Amgen (WO 2008118454, WO 2008118455, WO 2008118468) retain the H-bonding and hydrophobic interactions, while replacing the quinazolinone ring with a quinoline.

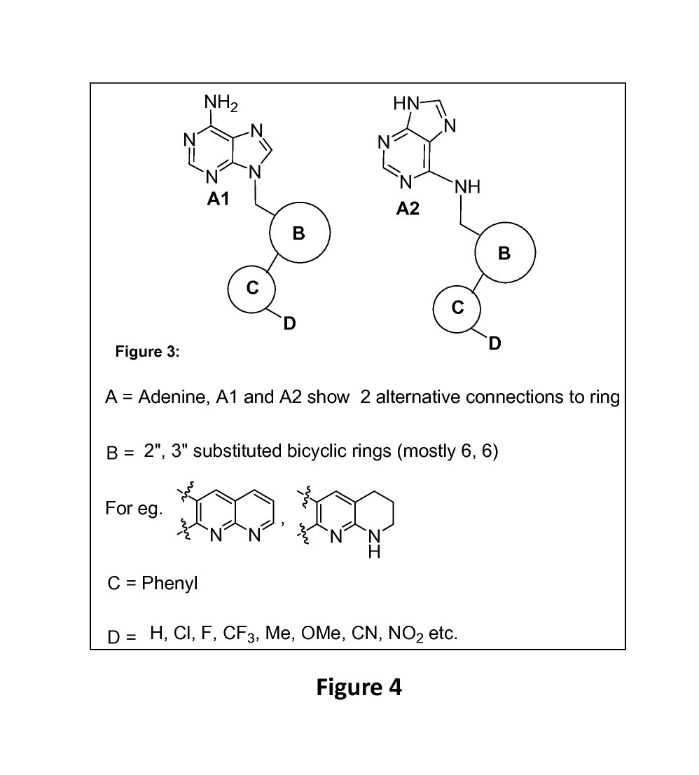

Experiments were designed to identify a novel quinazolinone replacement (B, FIG. 4) that maintains good interactions with the active site. Quinazolinones are notoriously insolubile. Therefore, a group that maintains favorable binding properties, while improving drug likeness relative to both the quinazolines and quinolines is desireable. Quinolines have a fairly basic heterocycle, which may contribute to p450 interaction or other toxicity. The B ring system that scored best in the initial evaluation was the less basic naphthyridine ring (7, FIG. 5). Although structurally similar to the Amgen series (WO 2008118454, WO 2008118455, WO 2008118468), the naphthyridine ring is not disclosed therein. Other B rings that scored well in the present assays are candidate lead compounds.

### Example 1

### Chemistry and Biology Studies

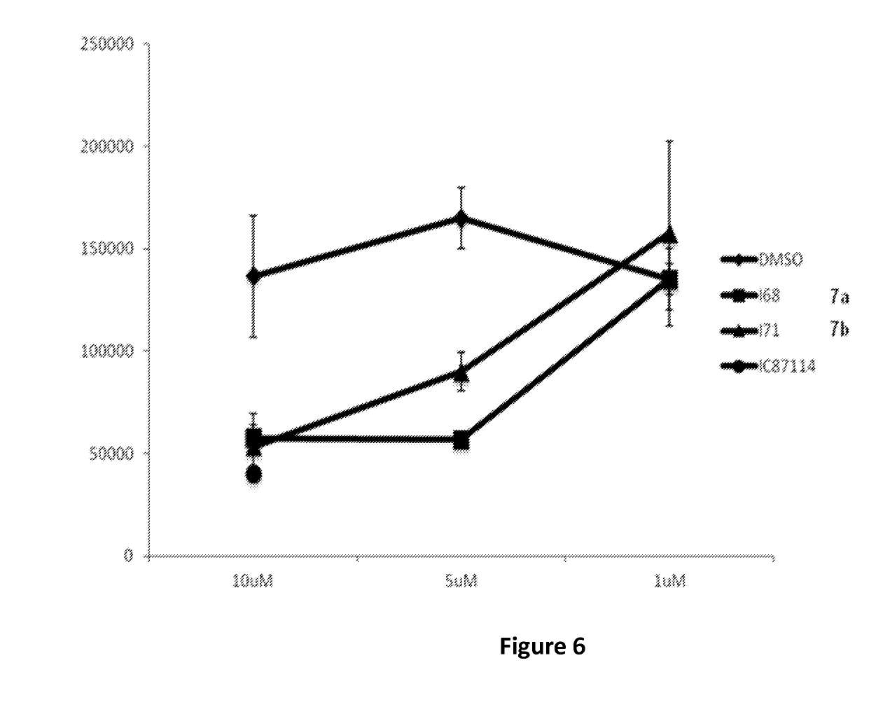

The preparation of the analogs is depicted in FIG. 5. The key intermediate 5 was not easily prepared using literature procedures on similar substrates. The challenge was the bromination step c. A successful bromination was dependent on the oxidation state of the heterocycle and was best achieved on the naphthyridone 3. With compound 4 in hand, aromatization and chlorination was accomplished with phosphorus oxychloride. Alkylation with adenine and Suzuki-Miyaura coupling afforded compounds 7 in good yields. The structures of intermediates and final compounds were unambiguously established by ¹H NMR and MS. Compounds 7a/7b were evaluated for activity in vitro activity against PI3K isoforms (7b: IC₅₀=1.3, δ; >100, α; >100, β; and 40 μM, γ) and influenza virus (FIG. 6) and they demonstrated similar potency and selectivity to IC87114.

### Example 2

### Novel PI3K p110d Inhibitors as Anti-Viral Agents

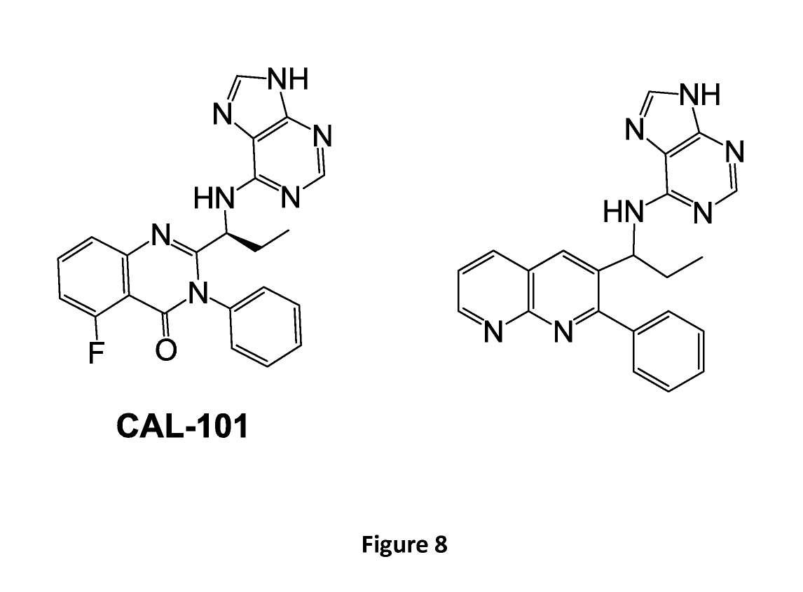

Experiments were designed to develop potent and safe inhibitors of p110δ that can be used as therapeutics against influenza virus infection. A model of the human p110δ active site was used to query multiple potential ligands by docking them into p110δ. The structures docked into the enzyme were chosen from two alternative connections to adenine (A1 and A2, FIG. 4), thirty-five 2″,3″-substituted bicyclic rings (mostly 6, 6), eighteen substituents on B, and twenty-five ortho substituents on C=Phenyl. The designed analogs maintained the H-bond donating/accepting pair of the adenine ring. The perpendicular aromatic rings with one ortho-substituent also appeared to be critical for efficient binding. Interactions of these aromatic rings with hydrophobic residues Trp760, Ile777, Pro758 and Met752 were operative. The goal was to identify a novel heterocycle (B, FIG. 4) that maintained good interactions with the active site. Quinazolinones contained in IC87114 and CAL-101 (FIG. 8) are notoriously insoluble, therefore a group that maintains favorable binding properties, while improving drug likeness relative to both the quinazolinones is of interest. Out of the multiple possible B ring systems evaluated, one that scored best in the evaluation was the weakly basic naphthyridine ring (7, FIG. 5). The optimal molecule was selected for synthesis and biological testing. An initial lead compound 7b was evaluated for activity in vitro activity against all class I PI3K isoforms (IC₅₀=1.3, p110δ; >100, p110α; >100, p110β; and 40 μM, p110γ) and influenza virus and it demonstrated similar potency and selectivity to IC87114, the first identified selective PI3K p110δ inhibitor.

Experiments were also designed to perform further structure-activity studies and in vitro and in vivo feasibility studies to demonstrate that the first-generation compounds can be modified to increase their potency against influenza virus, while maintaining their “drug like” features. The combination of PI3Kδ ligand/receptor structural information and docking studies are used to define the optimal chemical space surrounding prototype active compounds. The compounds identified by the methods disclosed herein can be evaluated initially for PI3K p110δ inhibition in vitro and once they meet defined criteria they can be advanced to selectivity testing against other PI3K isoforms and in vitro antiviral assays. Optimized lead structures can be examined for their broader kinase specificity, solubility, permeability, absorption, and metabolic stability.

The compounds can further be tested and evaluated in vivo against influenza virus infection of mice and can be further evaluated for their protective effect by examining influenza virus lung viral titers, pulmonary inflammation, morbidity and mortality.

Docking of Newly Proposed Compounds into Model of Human PI3K p110δ Using Induced Fit Based on CAL-101 to Predict Potency Enhancement

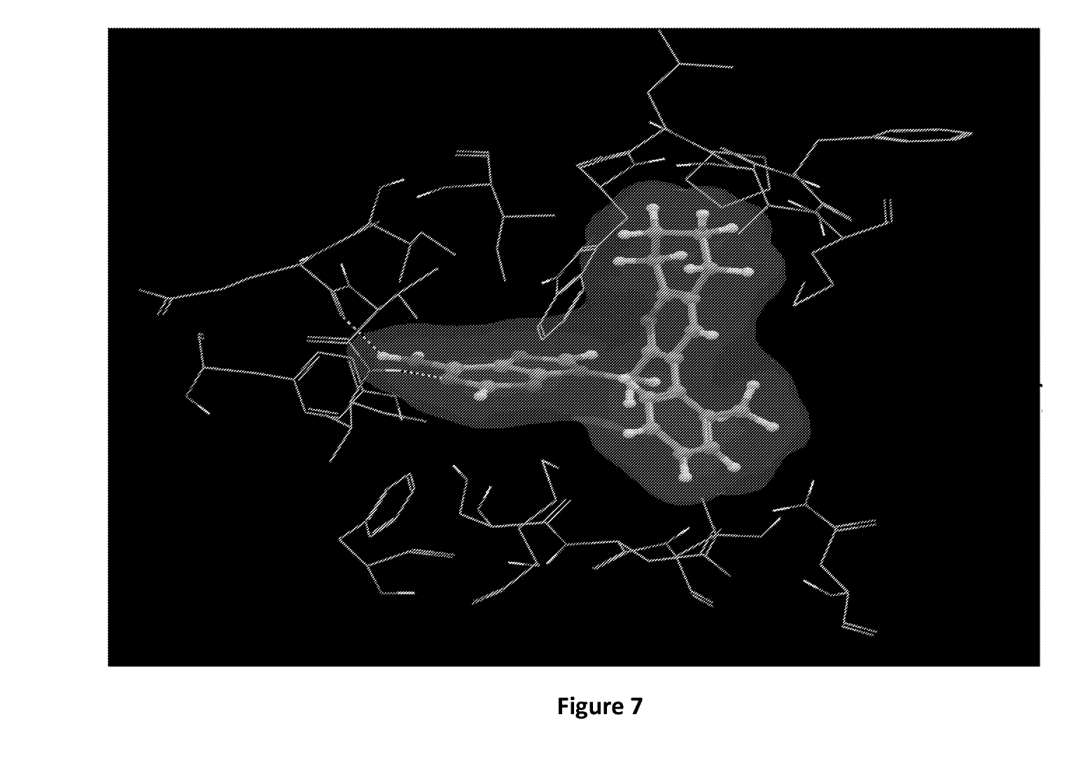

Docking studies were conducted to identify new compounds that inhibit p110δ. The receptor model disclosed in FIG. 7 was initially biased toward the binding configuration of IC87114, as shown in the x-ray pose. Therefore, it was not surprising that when CAL-101, a much more potent analog (δ IC₅₀=2 nM) (Lannutti et al., 2011, Blood 117(2):591-594), was docked, only a modest binding score was obtained. The receptor model was then optimized toward CAL-101 in order to define the optimum binding form for that potent analog.

The next experiments were designed to sample adenine isosteres, and substituents C and D (FIG. 4) to explore the newly optimized binding orientation. Once these groups are sampled, the preferred groups are combined with B-rings identified from previous docking experiments. In this iterative process, the range of possible series and structures for synthesis can be narrowed down. Then, analog clusters can be prepared from common key intermediates.

Next, experiments were designed to construct a human p110δ model. Maestro programs (Schrodinger Suite 2010) are used for molecular modeling on a LINUX platform. PI3K p110δ (2×38.pdb) containing IC87114 is imported and converted to a human homology model. A 16-angstrom region surrounding the active site is used for the docking studies. Protein Preparation Wizard caps and adds hydrogens to the enzyme. Potential inhibitors are modeled and minimized with Maestro. Ligprep added possible tautomers and ionization states to the proposed ligands. The Glide grids are generated with H-bonds to Val-828 (NH) and Glu-826 (═O) as constraints. Induced Fit Docking is used on a subset of ligands with Extra-Precision scoring. Lipinski's rules and calculated solubility, permeability, and oral absorption were monitored. Compounds are selected based on the desirable range.

### Design, Synthesize, and Test of New Compounds for Testing and Scale Up for In Vivo Evaluation

Experiments were designed to evaluate new compounds in an iterative process for activity in vitro at p110δ. It is desirable to identify a compound with IC₅₀of <100 nM. Prioritized compounds can be evaluated for selectivity (p110α, p110β, and p110γ vs. p110δ; >25 fold), antiviral activity in cells (IC₅₀<1 μM), selectivity relative to representative kinases; >100 fold), and desirable ADME properties in vitro.

Prototype optimization can be performed as follows. A prototype compound can be synthesized and tested for in vitro potency in vitro against PI3Kδ. Compounds that have greater potency than 1 μM, can then be evaluated for selectivity and anti-viral activity in vitro. If the series is appealing based on these results, a library of targets around that lead can be pursued. Targets for immediate synthesis include 7b with the A2 adenine orientation, and the derivative with the tetrahydronaphthyridine ring system (FIG. 4), which can be obtained by partial hydrogenation. An ethyl group can be incorporated on the methylene group connecting the B-ring to the A-2 ring to give target 8. These two changes converted M inhibitor IC87114 to the nM inhibitor CAL-101 (FIG. 8), which is also selective relative to other kinases (Lannutti et al., 2011, Blood 117(2):591-594). Variation of the phenyl substituent, the C-ring, and adenine isosteres can also be considered. Variation of the C-ring has yielded some of the highest docking scores.

Lead Optimization can be performed as follows. Select prototype molecules can be evaluated in various ADME and safety pharmacology assays in order to identify a Lead Candidate that meets most of the desired criteria. The minimal requirement is to identify a molecule that has adequate efficacy, selectivity, permeability, stability, and safety data to support investigation of the molecule in an in vivo efficacy model.

A PI3K assay can be used to evaluate the activity of the prototype molecule. Reaction Biology Corporation (RBC) Malvern, Pa. offers Class I PI3Ks (PI3Kα, PI3Kβ, PI3Kγ, and PI3Kδ). The PIP3 product is detected by displacement of biotin-PIP3 from an energy transfer complex consisting of Europium labeled anti-GST monoclonal antibody, a GST-tagged pleckstrin homology (PH) domain, biotinylated PIP3 and Streptavidin-Allophycocyanin (APC). Excitation of Europium in the complex results in an energy transfer to the APC and a fluorescent emission at 665 nm. The PIP3 product formed displaces biotin-PIP3 from the complex resulting in a loss of energy transfer and thus a decrease in signal.

An in vitro anti-viral assay can be used to evaluate the activity of the prototype molecule. A549 cells (ATTC) are plated at 5×10⁵ cells per well, infected with 0.1 MOI of PR8 virus for 2 hours and treated with the inhibitor. Virus is removed after 24 hours of infection; cells are washed with PBS, and harvested 48 hour later. Supernatants are tested in a plaque assay using MDCK cells, as previously described elsewhere herein.

Evaluate Promising PIK3 p110δ Leads in an Infection Model in Mice

Experiments were designed to identify a compound that reduces viral titers and morbidity, and increases survival relative to vehicle controls in an influenza infection model and increases potency as compared to IC87114.

An influenza virus infection model can be used to evaluate the activity of the compound in an infected animal model. Briefly, specific pathogen-free 8-12 week old C57BL/6 female mice can be purchased from Jackson Laboratories. All mice are maintained in AAALAC certified barrier facilities at Drexel University College of Medicine and experiments are performed after IACUC approval. Mice are anesthetized with avertin and infected intranasally with influenza virus.

The mice can be used to evaluate morbidity, viral titers and inflammation as a result of influenza infection. Mice are infected i.n. with a sublethal dose (3 TCID₅₀) of influenza virus A/PR/8/34 (H1N1) and weight loss is monitored daily for 20 days (n=9 mice per group). Uninfected animals serve as controls. Lung influenza viral titers are measured on days 3, 6 and 10 in groups of n=9 mice (3 experiments performed, with n=3 per group). Uninfected animals serve to establish baselines. To assess lung inflammation, cellular infiltration and cytokines are measured in the lungs. Lungs are harvested on days 3, 6 and 10 post-infection. The lungs are divided in 3 pieces of approximately equal weight. One piece can be saved in culture media, digested and used further for flow cytometry analysis, a second piece can be homogenized in PBS supplemented with protease inhibitors and cell supernatant can be frozen until ELISA cytokines are performed and a third piece can be homogenized in TRIzol and used for mRNA purification.

The weight of the total lung and of each piece can be measured before processing so that results can be presented per mg tissue. Cellular infiltrates can be determined by 10-12-color flow cytometry on digested lung tissue. Lung single cell suspensions can be counted and stained with fluorochrome-conjugated anti-mouse monoclonal antibodies that define lymphoid cell populations (CD3, CD4, CD8, CD19, NK1.1), granulocytes (GR-1), macrophages (CD11b, F4/80), dendritic cells (CD11c and CD11b) and activation markers (CD69 and CD25 for T cells, CD69 for NK cells, CD86 and MHC class II for B cells, dendritic cells and macrophages). Total numbers of cell populations can be extrapolated based on weight and presented as cells per lung. For each time point nine mice can be evaluated. The number and activation status of different cell populations in the lungs of animals can be compared. The piece of lung saved in TRIzol can be used for quantitation of proinflammatory cytokine mRNA by Real-Time PCR (RT-PCR) using commercially available validated primers for IFNα, IFNβ, IFNγ, TNFα, IL-1β, IL-18, IL-6, IL-10 and β-actin as a house keeping gene. As a baseline control, mRNA from uninfected mice can be used. Because cytokines can be regulated at the post-transcriptional level, experiments can be performed to quantitate in lung lysates for the amount of cytokine protein by using specific cytokine ELISAs for IFNα, IFNβ, IFNγ, TNFα, IL-β, IL-18, IL-6 and IL-10. As a baseline control, uninfected lungs can be used. The amount of cytokines determined by either RT-PCR or ELISA can be normalized to 100 mg tissue, based on the weight of the lung piece originally determined. Viral titers can be determined by RT-PCR on lung RNA isolated as discussed elsewhere herein. The viral load can be calculated using a standard curve based on viral cDNA from an influenza virus stock of known concentration. The viral load can be normalized to 100 mg tissue.

Survival studies can be performed to evaluate the activity of the compounds in vivo. For survival studies, mice are infected with 1 TCID₅₀ of H7N7 London virus strain (A/Equine/London/1416/73). Weight loss and survival is monitored on a daily basis. It has been determined that 1 TCID₅₀ of London influenza virus administered i.n. induces a drastic and rapid weight loss in the first 7 days of infection. Once mice lose 30% of their body weight, they are euthanized (animals have to be removed when weight loss is >30% according to IACUC regulations). Animals that lose >30% weight do not recover from influenza virus infection, therefore this does not alter true survival. Death is not an end point. Nine mice are infected per group. For uninfected controls, nine mice are used. Mouse weight is recorded daily. Mice are removed from the study when they lose 30% of their initial body weight or they become moribund. Mortality is recorded as the percentage of mice that had >30% weight loss and had to be euthanized.

### Example 3

### Discovery of Novel PI3K p110δ Inhibitors as Anti-Viral Agents

The emergence of drug resistance and the threat of pandemics have made critical the development of novel influenza virus therapies. The structure of murine p110δ bound to its selective ligand IC87114 (PI3K d IC₅₀=0.5 μM) is known and can be used for drug design. The murine structure is converted to a human homology model. The human model can be used to compare the interactions of known and proposed inhibitors to both p110δ isoforms.

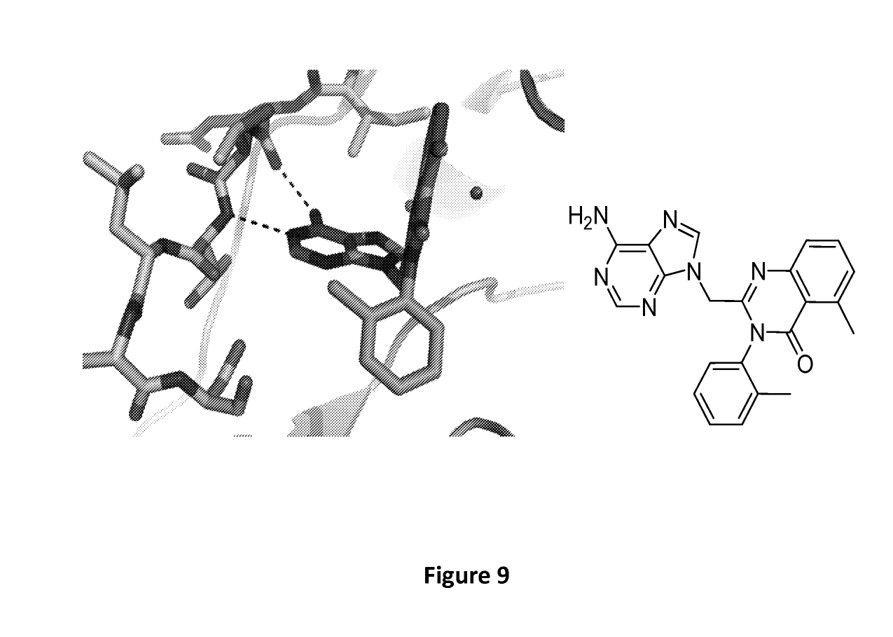

The analogs of the present invention maintain the H-bond donating/accepting pair of a heterocyclic ring, analogous to the adenine ring of IC87114 in the FIG. 9. The perpendicular aromatic rings with one ortho-substituent also appear to be critical for efficient binding. Interactions of these aromatic rings with hydrophobic residues Trp760, Ile777, Pro758 and Met752 are operative. A series of PI3K8 inhibitors from Amgen retain the H-bonding and hydrophobic interactions, while replacing the quinazolinone ring with a quinoline.

Experiments were designed to identify a novel quinazolinone replacement and bridging chain that maintains good interactions with the active site. Once appropriate candidates have been identified, prototypes can be synthesized. These inhibitors can screened initially for anti-viral activity in cell culture, and active inhibitors can followed up in p110δ and selectivity screens. The desirable target product profile is shown in Table 1.

TABLE 1
Target Product Profile

| | |
|---|---|
| Kinase Inhibition | IC₅₀ <10 nM, |
| Antiviral Activity in Cells | IC₅₀ <100 nM |
| Selectivity relative to related Kinases | 100-fold |
| Molecular Weight | <500 |
| Aqueous Solubility | >100 μg/mL |
| Log D | 1-3 |
| Permeability Caco-2 | High |
| Solution Stability | Stable |
| Metabolic Stability | t_{1/2} >40 min |
| CYP P₄₅₀ | IC₅₀ >10 μM |
| Protein Binding (human) | <90% |
| Bioavailability (rodent, non-rodent) | F >30% |
| Half life (iv; t_{1/2}, h) | >5 h |
| hERG | IC₅₀ >10 μM |
| CV Dog Telemetry | No QT prolongation |
| In vitro micronucleus | Neg. |
| Genotoxicity; Complete Ames | Neg. |

### Example 4

### Synthesis of 1,8-Naphthyridine Derivatives

The scheme for synthesizing 1,8-naphthyridine derivatives is as follows:

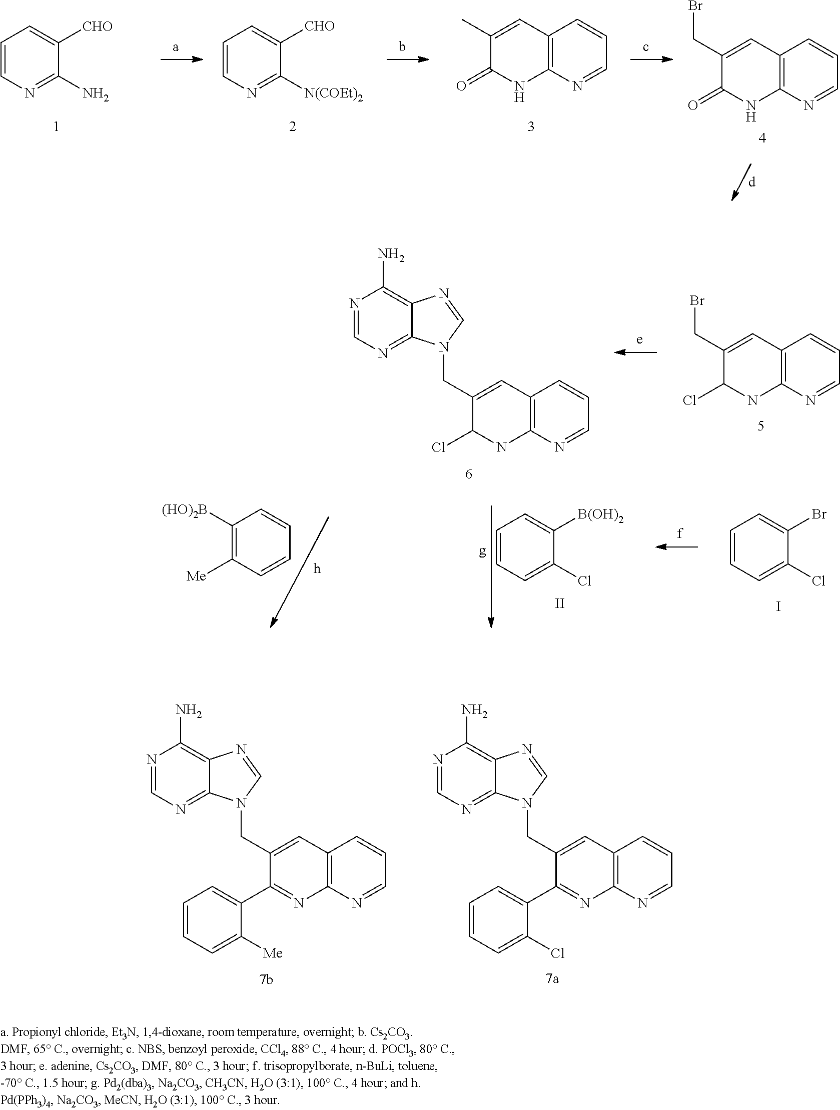

### Procedure (a):

Propionyl chloride (1.07 ml, 3 eq., 12. 28 mmol) and triethylamine (1.7 ml, 3 eq., 12. 28 mmol) were added drop wise to a solution of 2-amino-nicotinaldehyde (0.5 g, 4.1 mmol) in 1,4-dioxane (15 ml) with stirring at 0° C. under N₂. The resulting mixture formed a yellow suspension. The yellow suspension was stirred at room temperature overnight. TLC (thin layer chromatography) in 30% EtOAc and hexane showed that the product traveled above the starting material, demonstrating a completion of the reaction. The solvent was evaporated in vacuo. The resulting residue was taken up in ethyl acetate. The organic phase was washed with water and dried over Na₂SO₄. The organic phase was then concentrated in vacuo

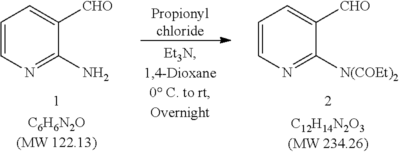

### Procedure (b):

Cs₂CO₃ (2.67 g, 8.2 mmol, 2 eq) was added to a suspension of yellow crude compound 2 in DMF under N₂. The resulting mixture was heated at 65° C. overnight. TLC in 40% EtOAc and Hexane showed that the product traveled just below the starting material and demonstrates completion of the reaction. Reaction mixture was cooled to room temperature and DMF was evaporated. Ice water was then added to the residue. The precipitates were filtered. The filter cake was washed with cold water and ethyl acetate to give a white solid in 81% (532 mg) yield in two steps. ¹H NMR (CDCl₃): δ ˜11.9 (br, 1H, exchanged upon addition of D₂O), 8.61 (br d, J=3.6 Hz, 1H), 7.85 (dd, J=7.8 Hz, 1H), 7.57 (d, J=1.2 Hz, 1H), 7.19 (dd, J=2.2 Hz, J=5.1 Hz, 1H), 2.27 (s, 3H); wherein “br” is broad. .LC-MS: RT=2.47 min, m/z=161.08.

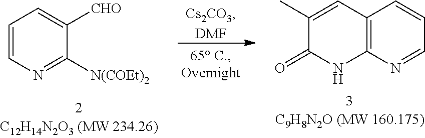

### Procedure (c):

NBS (666 mg, 1.2 eq., 3.75 mmol) and dibenzoyl peroxide (113.11 mg, 0.47 mmol, 0.15 eq.) were added to a suspension of compound 3 (0.5 g, 3.12 mmol) in CCl₄ (25 ml). The resulting mixture was stirred at 90° C. for 4-5 hours. Solvent was evaporated. Upon addition of methanol to the reaction mixture, precipitates were obtained. The precipitates were further washed with methanol to give a white solid in 80% yield (600 mg). ¹H NMR (CDCl₃): δ ˜12.1 (br, 1H, exchanged upon addition of D₂O), 8.70 (d, J=3.3 Hz, 1H), 7.95 (dd, J=1.5 Hz, J=7.8 Hz, 1H), 7.92 (br s, 1H), 7.25 (1H), 4.55 (s, 2H). .LC-MS: RT=2.92 min, m/z=238.9, 240.9.

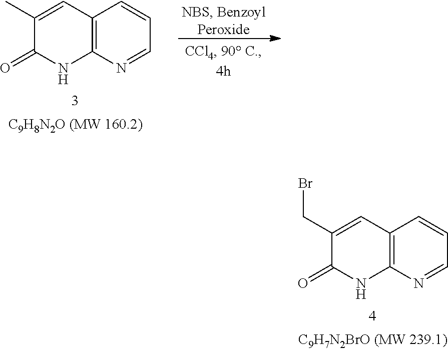

### Procedure (d):

500 mg of compound 4 was heated at 88° C. with 5 ml of POCl₃ for 3-4 hours. The reaction mixture was cooled to room temperature then poured into ice water. The resulting solution was neutralized to pH 8 using 40% NaOH solution. White precipitates were filtered and washed with cold water and dried. Yield=400 mg (74.3%).

TLC in 1:1 EtOAc and hexane showed no difference in product and starting material. However, after workup and purification, HNMR and LCMS showed the presence of product. Other solvent systems were also used but none of them could separate the product from the starting material. ¹H NMR (CDCl₃): δ 9.15 (dd, J=1.8 Hz, J=4.2 Hz, 1H), 8.37 (s, 1H), 8.25 (dd, J=1.8 Hz, J=8.4 Hz, 1H), 7.56 (dd, J=3.9 Hz, J=6.6 Hz, 1H), 4.88 (br s, 2H). .LC-MS: RT=3.38 min, m/z=258.9, 260.9.

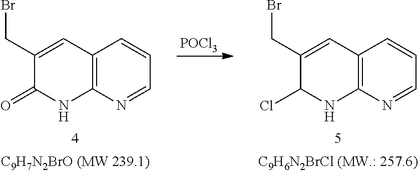

### Procedure (e):

A mixture of compound 5 (100 mg, 0.386 mmol), Cs₂CO₃ (136.2 mg, 1 eq., 0.386 mmol) and adenine (57.33 mg, 1.1 eq., 0.43 mmol) in DMF was stirred at room temperature for 2 hours under nitrogen. TLC in 1:1 EtOAc and Hexane and in 10% MeOH and DCM showed that no reaction occurred. Therefore, the reaction was heated at 80° C. After 3 hours, the TLC showed complete conversion. Solvent was removed and the compound was purified using 2%-7% MeOH, Dichloromethane.

Yield=100 mg (82.6%). ¹H NMR (DMSO-d): δ 9.09 (dd, J=1.8 Hz, J=3.9 Hz, 1H), 8.52 (dd, J=2.1 Hz, J=10.2 Hz, 1H), 8.28 (s, 1H), 8.12 (d, J=4.8 Hz, 2H), 7.67 (dd, J=4.4 Hz, J=8.4 Hz, 1H), 7.32 (br s, 2H), 5.66 (s, 2H). LC-MS: RT=2.17 min, m/z=312.0, 314.0.

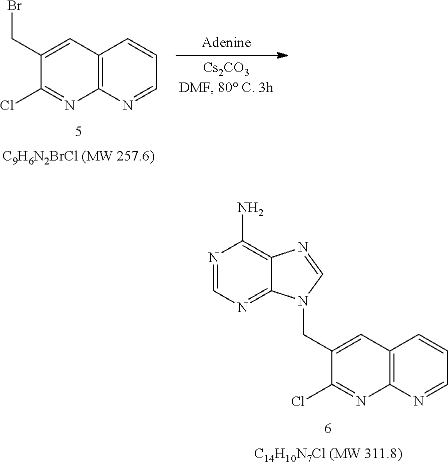 [SMILES: Clc1nc2ncccc2cc1CBr ; Nc1ncnc2c1ncn2Cc1cc2cccnc2nc1Cl]

### Procedure (f):

n-BuLi (2.5 M in n-Hexane, 2.5 ml, 1.2 eq., 6.3 mmol) was added dropwise to a solution of Bromo chloro benzene (I) (1 g, 0.6 ml, 5.2 mmol) and Triisopropylborate (1.44 ml, 1.2 eq., 6.27 mmol) in Toluene and THF (4:1, 10 ml) under nitrogen at −70° C. over 1 hour. The reaction mixture was stirred for an additional 0.5 hour while the temperature was held at −70° C. The reaction mixture was allowed to warm to −20° C., before a 2 N HCl solution (5 ml) was added to the reaction mixture. When the reaction mixture reached room temperature, it was extracted with Dichloromethane. Combined organic phase was dried and evaporated to give a white solid, which was recrystallized from MeCN with a yield of 98% (800 mg).

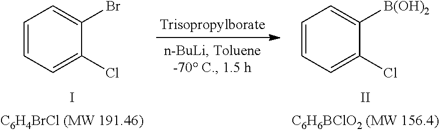

### Procedure (g):

A mixture of compound 6 (15 mg, 0.05 mmol), 2-Chloro phenyl boronic acid (8.3 mg, 1.1 eq., 0.053 mmol), Pd₂(dba)₃ (2.8 mg, 0.05 eq., 0.0024 mmol), Na₂CO₃ (25.5 mg, 5 eq., 0.24 mmol) in MeCN and water (3:1, 2 ml) was heated at 100° C. under nitrogen atmosphere for 30 min. TLC in 7% MeOH and DCM showed no separation between starting material and product. TLC showed that the product appeared just above the starting material based on the color difference. Water was added to the reaction mixture and the product was extracted with EtOAc. The combined organic layers were dried over Na₂SO₄ and filtered. The organic layers were then concentrated and purified by column chromatography using 1%-7% MeOH and DCM to afford compound 7a. Yield=˜75%, 5 mg˜90% pure, 10 mg has little impurity in it. ¹H NMR (CDCl₃): δ 9.16 (dd, J=2.1 Hz, J=4.2 Hz, 1H), 8.32 (s, 1H), 8.20 (dd, J=1.5 Hz, J=7.8 Hz, 1H), 8.14 (s, 1H), 7.55-7.33 (m, 5H), 5.58 (br s, 2H), 5.49 (s, 1H), 5.44 (s, 1H). LC-MS: RT=2.37 min, m/z=388.0, 390.0.

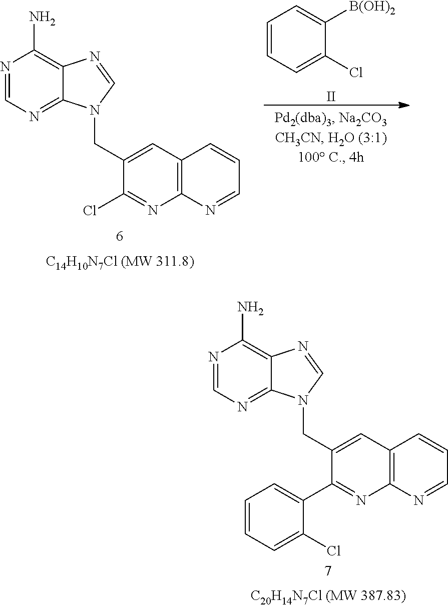

### Procedure (h):

A mixture of compound 6 (50 mg, 0.16 mmol), 2-Methyl phenyl boronic acid (24 mg, 1.1 eq., 0.18 mmol), Pd₂(dba)₃ (9.3 mg, 0.05 eq., 0.008 mmol) Na₂CO₃ (85.2 mg, 5 eq., 0.24 mmol) in MeCN and water (3:1, 4 ml) was heated at 100° C. under nitrogen atmosphere for 3 hour. TLC in 2.5-3% MeOH and DCM showed that the product traveled above but very close to the starting material. After running the TLC several times in the same solvent system, the product and the starting material showed little separation. Solvent was evaporated and the compound was purified by column chromatography using 1-5% MeOH and DCM to give a white solid 7b in 93% yield (55 mg). ¹H NMR (CD₃OD): δ 9.09 (1H), 8.49 (1H), 8.41 (dd, J=2.1 Hz, J=8.1 Hz, 1H), 8.06 (1H), 7.68 (dd, J=4.2 Hz, J=8.1 Hz, 1H), 7.50 (1H), 7.38-7.27 (m, 4H), 5.46 (m, 2H), 1.93 (s, 3H). LC-MS: RT=2.27 min, m/z=368.0.

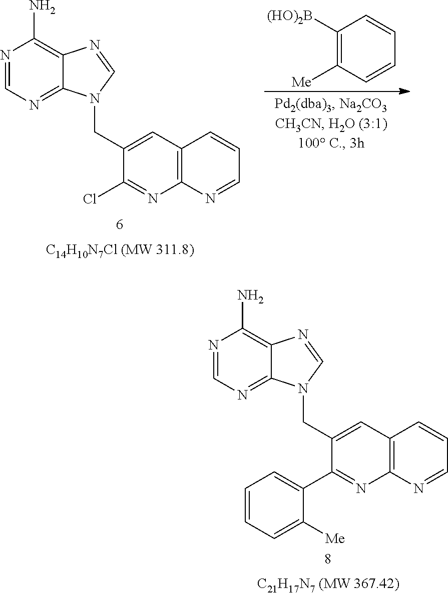 [SMILES: Nc1ncnc2c1ncn2Cc1cc2cccnc2nc1Cl ; Cc1ccccc1B(O)O ; Cc1ccccc1-c1nc2ncccc2cc1Cn1cnc2c(N)ncnc21]

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of Formula (I), or a salt thereof: 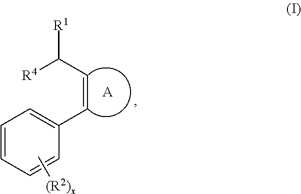
wherein in (I):
R¹ is selected from the group consisting of:
6-amine-9H-purin-9-yl, (9H-purin-6-yl)amino, 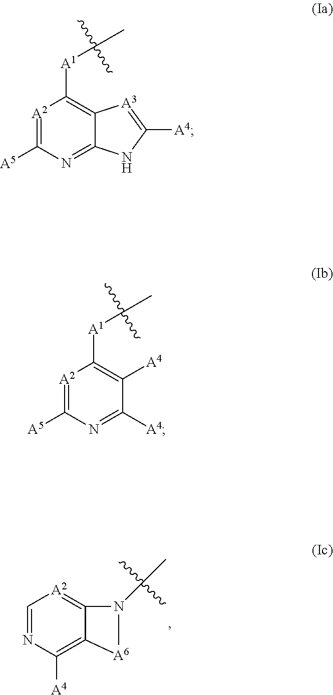
wherein in (Ia), (Ib) and (Ic):
A¹ is N(R⁸), O or S;
A² and A³ are independently C(R⁸) or N;
each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘNHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;
ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not: 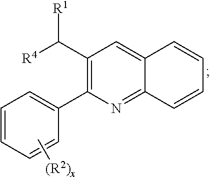
each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘNHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;
R⁴ is H, —C₁-C₆ alkyl, —C₁-C₆ fluoroalkyl or —C₁-C₆ heteroalkyl;
each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;
R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;
L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃alkylene)ₘ-NH—(C₁-C₃ alkylene)ₘ-;
x is 0, 1, 2 or 3; and,
each occurrence of m is independently 0, 1 or 2.

2. The compound of claim 1, wherein ring A is selected from the group consisting of pyridine, pyrimidine, quinoline, isoquinoline, 1,8-naphthyridine, 1,7-naphthyridine, 1,6-naphthyridine, 1,5-naphthyridine, pyrido[2,3-c]-pyridazine, pyrido[2,3-d]-pyrimidine, pyrido[2,3-b]-pyrazine, and 1,2,3,4-tetrahydro-1,8-naphthyridine.

3. (canceled)

4. (canceled)

5. The compound of claim 1, wherein the compound of Formula (I) is selected from the group consisting of:

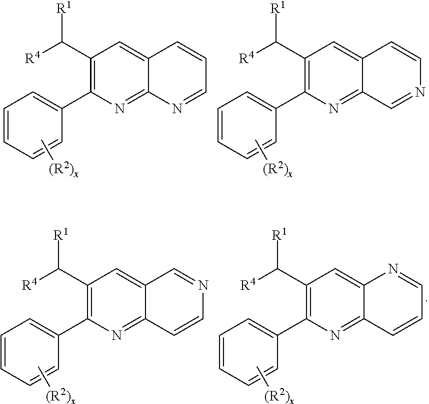

6. (canceled)

7. (canceled)

8. (canceled)

9. The compound of claim 1, wherein the compound of Formula (I) is selected from the group consisting of:
9-((6-(2-chlorophenyl)quinolin-7-yl)methyl)-9H-purin-6-amine;
9-((6-(2-methylphenyl)quinolin-7-yl)methyl)-9H-purin-6-amine;
9-((2-(2-chlorophenyl)-1,8-naphthyridin-3-yl)methyl)-9H-purin-6-amine;
9-((2-(2-methylphenyl)-1,8-naphthyridin-3-yl)methyl)-9H-purin-6-amine;
N-(1-(2-phenyl-1,8-naphthyridin-3-yl)propyl)-9H-purin-6-amine;
a salt thereof, and any combinations thereof.

10. (canceled)

11. (canceled)

12. (canceled)

13. A method of inhibiting replication of a virus in a cell, the method comprising contacting the cell with an inhibitor of PI3K p110 delta, wherein the contacting inhibits PI3K110 delta in the cell, thereby inhibiting replication of the virus in the cell, wherein the inhibitor is a compound of Formula (I) or a salt thereof: 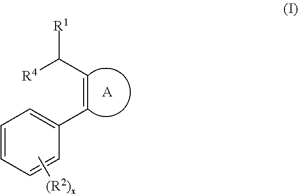
wherein in (I):
R¹ is selected from the group consisting of:
6-amine-9H-purin-9-yl, (9H-purin-6-yl)amino, 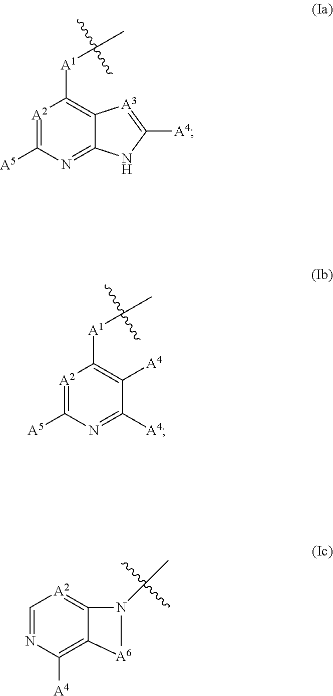
wherein in (Ia), (Ib) and (Ic):
A¹ is N(R⁸), O or S;
A² and A³ are independently C(R⁸) or N;
each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘNHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;
ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not: 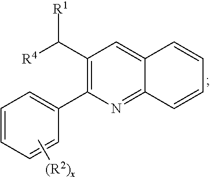
each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘNHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;
R⁴ is H, —C₁-C₆ alkyl, —C₁-C₆ fluoroalkyl or —C₁-C₆ heteroalkyl;
each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;
R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;
L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃alkylene)ₘ-NH—(C₁-C₃ alkylene)ₘ-;
x is 0, 1, 2 or 3; and,
each occurrence of m is independently 0, 1 or 2.

14. The method of claim 13, wherein the virus is influenza.

15. The method of claim 13, wherein ring A is selected from the group consisting of pyridine, pyrimidine, quinoline, isoquinoline, 1,8-naphthyridine, 1,7-naphthyridine, 1,6-naphthyridine, 1,5-naphthyridine, pyrido[2,3-c]-pyridazine, pyrido[2,3-d]-pyrimidine, pyrido[2,3-b]-pyrazine, and 1,2,3,4-tetrahydro-1,8-naphthyridine.

16. (canceled)

17. (canceled)

18. The method of claim 13, wherein the compound of Formula (I) is selected from the group consisting of:

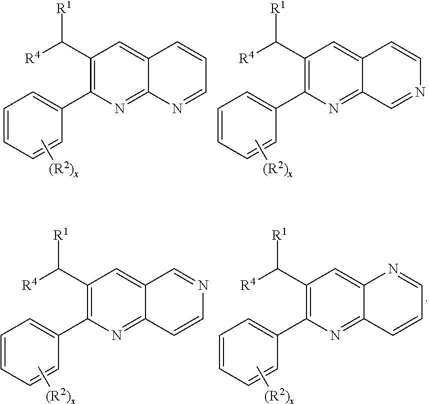

19. (canceled)

20. (canceled)

21. (canceled)

22. The method of claim 13, wherein the compound of Formula (I) is selected from the group consisting of:
9-((6-(2-chlorophenyl)quinolin-7-yl)methyl)-9H-purin-6-amine;
9-((6-(2-methylphenyl)quinolin-7-yl)methyl)-9H-purin-6-amine;
9-((2-(2-chlorophenyl)-1,8-naphthyridin-3-yl)methyl)-9H-purin-6-amine;
9-((2-(2-methylphenyl)-1,8-naphthyridin-3-yl)methyl)-9H-purin-6-amine;
N-(1-(2-phenyl-1,8-naphthyridin-3-yl)propyl)-9H-purin-6-amine;
a salt thereof, and any combinations thereof.

23. (canceled)

24. (canceled)

25. (canceled)

26. (canceled)

27. A method of inhibiting pathogenesis of a virus in a mammalian cell, the method comprising contacting the cell with a therapeutically effective amount of an inhibitor of PI3K p110 delta, wherein the contacting inhibits PI3K p110 delta in the cell, thereby inhibiting pathogenesis of the virus in the mammalian cell, wherein the inhibitor is a compound of formula (I) or a salt thereof: 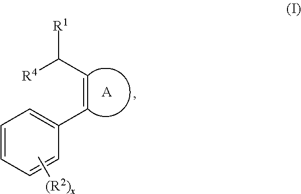
wherein in (I):
R¹ is selected from the group consisting of:
6-amine-9H-purin-9-yl, (9H-purin-6-yl)amino, 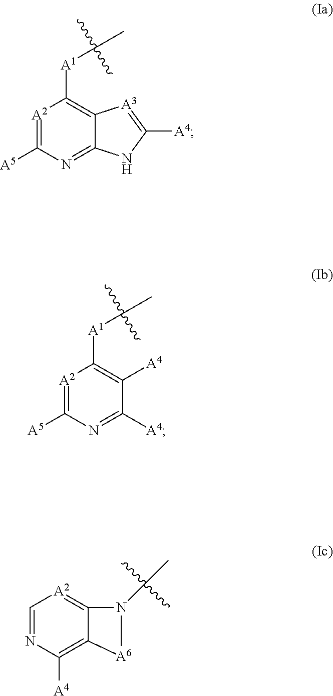
wherein in (Ia), (Ib) and (Ic):
A¹ is N(R⁸), O or S;
A² and A³ are independently C(R⁸) or N;
each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘNHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;
ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not: 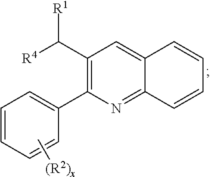
each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘNHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;
R⁴ is H, —C₁-C₆ alkyl, —C₁-C₆ fluoroalkyl or —C₁-C₆ heteroalkyl;
each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;
R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;
L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃alkylene)ₘ-NH—(C₁-C₃ alkylene)ₘ-;
x is 0, 1, 2 or 3; and,
each occurrence of m is independently 0, 1 or 2.

28. The method of claim 27, wherein the virus is influenza.

29. The method of claim 27, wherein ring A is selected from the group consisting of pyridine, pyrimidine, quinoline, isoquinoline, 1,8-naphthyridine, 1,7-naphthyridine, 1,6-naphthyridine, 1,5-naphthyridine, pyrido[2,3-c]-pyridazine, pyrido[2,3-d]-pyrimidine, pyrido[2,3-b]-pyrazine, and 1,2,3,4-tetrahydro-1,8-naphthyridine.

30. (canceled)

31. (canceled)

32. The method of claim 27, wherein the compound of Formula (I) is selected from the group consisting of:

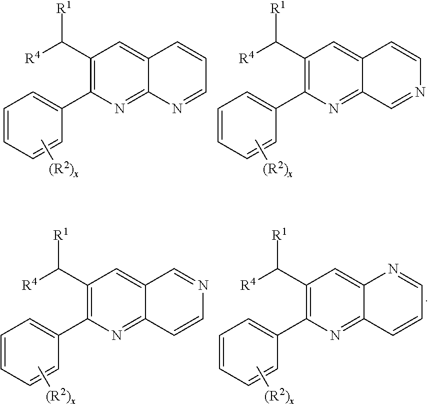

33. (canceled)

34. (canceled)

35. (canceled)

36. The method of claim 27, wherein the compound of Formula (I) is selected from the group consisting of:
9-((6-(2-chlorophenyl)quinolin-7-yl)methyl)-9H-purin-6-amine;
9-((6-(2-methylphenyl)quinolin-7-yl)methyl)-9H-purin-6-amine;
9-((2-(2-chlorophenyl)-1,8-naphthyridin-3-yl)methyl)-9H-purin-6-amine;
9-((2-(2-methylphenyl)-1,8-naphthyridin-3-yl)methyl)-9H-purin-6-amine;
N-(1-(2-phenyl-1,8-naphthyridin-3-yl)propyl)-9H-purin-6-amine;
a salt thereof, and any combinations thereof.

37. (canceled)

38. (canceled)

39. (canceled)

40. (canceled)

41. A method of treating or preventing infection by a virus in a mammal in need thereof, the method comprising administering a therapeutically effective amount of an inhibitor of phosphoinositide 3 kinase (PI3K) isoform p110 delta to the mammal, wherein the inhibitor interferes with PI3K p110 delta activation and replication of the virus in the mammal, thereby treating or preventing the infection in the mammal, wherein the inhibitor is a compound of formula (I) or a salt thereof: 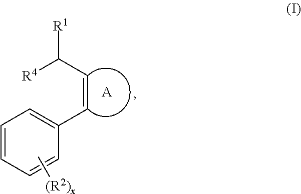
wherein in (I):
R¹ is selected from the group consisting of:
6-amine-9H-purin-9-yl, (9H-purin-6-yl)amino, 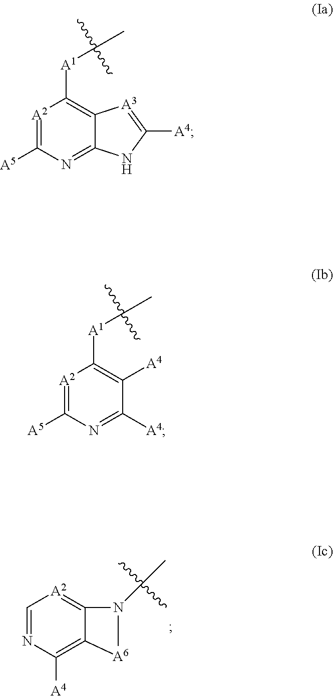
wherein in (Ia), (Ib) and (Ic):
A¹ is N(R⁸), O or S;
A² and A³ are independently C(R⁸) or N;
each occurrence of A⁴ and A⁵ is independently selected from the group consisting of H, F, Cl, Br, I, —CF₃, —CN, —NO₂, -(L)ₘ-OR⁸, -(L)ₘ-SR⁹, -(L)ₘ-S(═O)R⁹, -(L)ₘ S(═O)₂R⁹, -(L)ₘ-NHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘNHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl; and
A⁶ is —C(A⁴)═C(A⁵)-, —C(A⁴)═N—, —N═C(A⁴)-, —C(A⁴)═C(A⁵)-C(═O)— and —C(═O)—C(A⁴)═C(A⁵)-;
ring A is a monocyclic or bicyclic aryl ring, or a monocyclic or bicyclic heteroaryl ring, wherein the aryl or heteroaryl ring is optionally substituted with 0-3 substituents selected from R³, with the proviso that the compound of formula (I) is not: 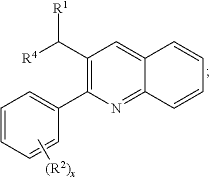
each occurrence of R² and R³ is independently selected from the group consisting of F, Cl, Br, I, —CF₃, —CN, —NO₂, -(L)ₘ-OR⁸, -(L)ₘ-SR⁹, -(L)ₘ-S(═O)R⁹, -(L)ₘ-S(═O)₂R⁹, -(L)ₘNHS(═O)₂R⁹, -(L)ₘ-C(═O)R⁹, -(L)ₘ-OC(═O)R⁹, -(L)ₘCO₂R⁸, -(L)ₘ-OCO₂R⁸, -(L)ₘ-CH(R⁸)₂, -(L)ₘ-N(R⁸)₂, -(L)ₘ-C(═O)N(R⁸)₂, -(L)ₘ-OC(═O)N(R⁸)₂, -(L)ₘ-NHC(═O)NH(R⁸), -(L)ₘ-NHC(═O)R⁹, -(L)ₘ-NHC(═O)OR⁹, -(L)ₘ-C(OH)(R⁸)₂, -(L)ₘC(NH₂)(R⁸)₂, —C₁-C₆ alkyl, —C₁-C₆ fluoroalkyl and —C₁-C₆ heteroalkyl;
R⁴ is H, —C₁-C₆ alkyl, —C₁-C₆ fluoroalkyl or —C₁-C₆ heteroalkyl;
each R⁸ is independently, at each occurrence, H, C₁-C₆ alkyl, C₁-C₆ fluoroalkyl, C₁-C₆ heteroalkyl, C₃-C₁₀ cycloalkyl, C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄alkyl(heteroaryl), and wherein the alkyl, heteroalkyl, cycloalkyl, heterocycloalkyl, aryl and heteroaryl is optionally substituted with 0-5 substituents selected from R²; or two R⁸ groups attached to the same N or C atom are taken together with the N or C atom to which they are attached to form an optionally substituted C₂-C₁₀ heterocycloalkyl or C₃-C₁₀ heterocycloalkyl, wherein the ring optionally comprises a moiety selected from O, C═O, S(O)ₘ, NR⁴S(O)ₘ, NR⁴(C═O) or N—R⁴, and wherein the ring is optionally substituted with 0-5 substituents selected from R²;
R⁹ is C₁-C₆ alkyl, C₁-C₆ fluoroalkyl, C₁-C₆ heteroalkyl, C₃-C₁₀ cycloalkyl, a C₂-C₁₀ heterocycloalkyl, aryl, heteroaryl, —C₁-C₄ alkyl-(C₃-C₁₀ cycloalkyl), —C₁-C₄ alkyl-(C₂-C₁₀ heterocycloalkyl), —C₁-C₄ alkyl-(aryl), or —C₁-C₄ alkyl-(heteroaryl), and wherein the alkyl, heteroalkyl, cycloalkyl, heterocycloalkyl, aryl or heteroaryl ring is optionally substituted with 0-5 substituents selected from R²;
L is independently at each occurrence a bivalent radical selected from —(C₁-C₃alkylene)ₘ-, —(C₃-C₇ cycloalkylene), —(C₁-C₃ alkylene)ₘ-O—(C₁-C₃ alkylene)ₘ-, or —(C₁-C₃alkylene)ₘ-NH—(C₁-C₃ alkylene)ₘ-;
x is 0, 1, 2 or 3; and,
each occurrence of m is independently 0, 1 or 2.

42. The method of claim 41, wherein the virus is influenza.

43. The method of claim 41, wherein ring A is selected from the group consisting of pyridine, pyrimidine, quinoline, isoquinoline, 1,8-naphthyridine, 1,7-naphthyridine, 1,6-naphthyridine, 1,5-naphthyridine, pyrido[2,3-c]-pyridazine, pyrido[2,3-d]-pyrimidine, pyrido[2,3-b]-pyrazine, and 1,2,3,4-tetrahydro-1,8-naphthyridine.

44. (canceled)

45. (canceled)

46. The method of claim 41, wherein the compound of Formula (I) is selected from the group consisting of:

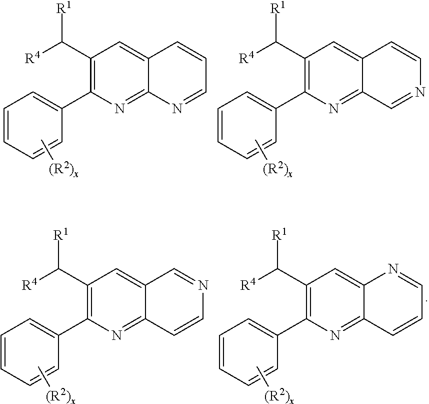

47. (canceled)

48. (canceled)

49. (canceled)

50. The method of claim 41, wherein the compound of Formula (I) is selected from the group consisting of:
9-((6-(2-chlorophenyl)quinolin-7-yl)methyl)-9H-purin-6-amine;
9-((6-(2-methylphenyl)quinolin-7-yl)methyl)-9H-purin-6-amine;
9-((2-(2-chlorophenyl)-1,8-naphthyridin-3-yl)methyl)-9H-purin-6-amine;
9-((2-(2-methylphenyl)-1,8-naphthyridin-3-yl)methyl)-9H-purin-6-amine;
N-(1-(2-phenyl-1,8-naphthyridin-3-yl)propyl)-9H-purin-6-amine;
a salt thereof, and any combinations thereof.

51. The method of claim 41, wherein the composition further comprises at least one anti-influenza drug or immunomodulator.

52. (canceled)

53. (canceled)

54. (canceled)

55. (canceled)
